(19) United States
(12) Patent Application Publication
(10) Pub. No.: US 2009/0012019 A1
(43) Pub. Date: Jan. 8, 2009

(54) Methods and Compositions Related to TR4

(76) Inventors: Chawnshang Chang, Pittsford, NY (US); Yifen Lee, Rochester, NY (US)

Correspondence Address: Ballard Spahr Andrews & Ingersoll, LLP, SUITE 1000, 999 PEACHTREE STREET, ATLANTA, GA, 30309-3915, US

(21) Appl. No.: 11/795,788

(22) PCT Filed: Jan. 20, 2006
(86) PCT No.: PCT/US06/02448

§ 371 Date: Aug. 25, 2008

Related U.S. Application Data
(60) Provisional application No. 60/645,466, filed on Jan. 20, 2005.

(51) Int. Cl. A61K 31/7105; G01N 33/53; A61P 43/00

(52) U.S. Cl. 514/44; 800/3; 435/40.52; 435/29

(57) ABSTRACT

Disclosed are compositions and methods related to TR4 and aging.

This application claims the benefit of U.S. Provisional Application No. 60/645,466, filed on Jan. 20, 2005, which is incorporated by reference herein in its entirety.

This application was made with government support under federal grants NIH U19 DK62434 awarded by the NIH. The Government has certain rights to this invention.

### I. SUMMARY

In accordance with the purposes of this invention, as embodied and broadly described herein, the disclosed compositions and methods, in one aspect, relates to TR4 and interactions between these molecules.

It is to be understood that both the foregoing general description and the following detailed description are exemplary and explanatory only and are not restrictive.

### II. BRIEF DESCRIPTION OF THE DRAWINGS

The accompanying drawings, which are incorporated in and constitute a part of this specification, illustrate several embodiments.

FIG. 1 shows the appearance of TR4KO and age matched wild type mice at 6 months of age. Greasy skin, dropping eye lids, long nails, and hunchback were seen in 6-month-old TR4 KO female mice.

FIG. 2 shows the mitochondria abnormality in TR4^{-/-} muscle trichrome staining shows ragged red fibers in 6-month old TR4^{-/-} soleus muscle, but not in TR4^{+/+}. Electric microscopic analyses on the same TR4^{-/-} soleus muscle display an abnormal proliferation of mitochondria accumulation at the side of muscle fiber.

FIG. 3 Skeletal abnormalities in aging TR4^{-/-} mice. Radiograph of 3 month and 6 month TR4^{-/-} and TR4^{+/+} mice. TR4^{-/-} mice display curvature of the spinal column (kyphosis), decreased BMD in both male (N=5)(B), and female (N=5) (C) mice.

FIG. 4 shows ovarian dysfunction in TR4^{-/-} female. FIG. 4A shows the female reproductive gross outlook of 3 month and 6 month TR4^{-/-} and TR4^{+/+} mice. FIG. 4B shows ovary histology analyses from TR4^{+/+} control female and TR4^{-/-} at 6 month old. Ovaries from TR4^{-/-} were small and contain immature preantral (PA) and antral (A) follicles; absence of corpora lutea (CL), and pre-ovulatory (PO) follicles compared to TR4^{+/+} female.

FIG. 5 shows the growth retardation and G2 arrest in KO MEF cells. The MEF cells for all genotypes were obtained from the same litter of 14 day embryos. After the 4th passage, the cell growth was determined on day 2, 4, and 6 by MTT assay, and cell cycle profiles were analyzed by flow cytometry.

FIG. 6 shows stress-induced TR4 expression. Low glucose induced TR4 expression. Mice dermis derived fibroblast cells were cultured with 1,000 mg/L D-glucose for 24 hrs, and cells were harvested 24 hrs later, then total RNA were extracted and TR4 mRNA expression levels were examined by RT-RCR (A), and Real-Time Q-PCR (B).

FIG. 7 shows TR45′-promoter analysis. A. Illustration of the putative transcriptional factors located in the TR45′-promoter. B. The basal transcriptional level tests in TR45′-promoter containing-luciferase. Serial deletions of TR45′-promoter have been constructed according to the available enzymes and then transfected into CV-1 cells. The basal transcriptional activities were assayed by luciferase.

FIG. 8 shows two clones of TR4 RNAi suppress TR4-mediated TR4RE-luc activity. CV-1 cells were co-transfected with pCMX-TR4, hTR4-siRNA 1-4, and 2-9 and TR4RE/ApoE (HCR-1-Luc) with different ratios as indicated, and then luciferase activities were measured 48 hr after transfection.

FIG. 9 shows a map of mtDNA genome. The positions of direct repeats are indicated in relation to the D-loop region and light strand origin of replication.

FIG. 10 shows a comparison of TR4 wild-type (wt) and knock-out (KO) mice. While both have similar albumin levels, the TR4 knock outs have gray hair and a smaller size.

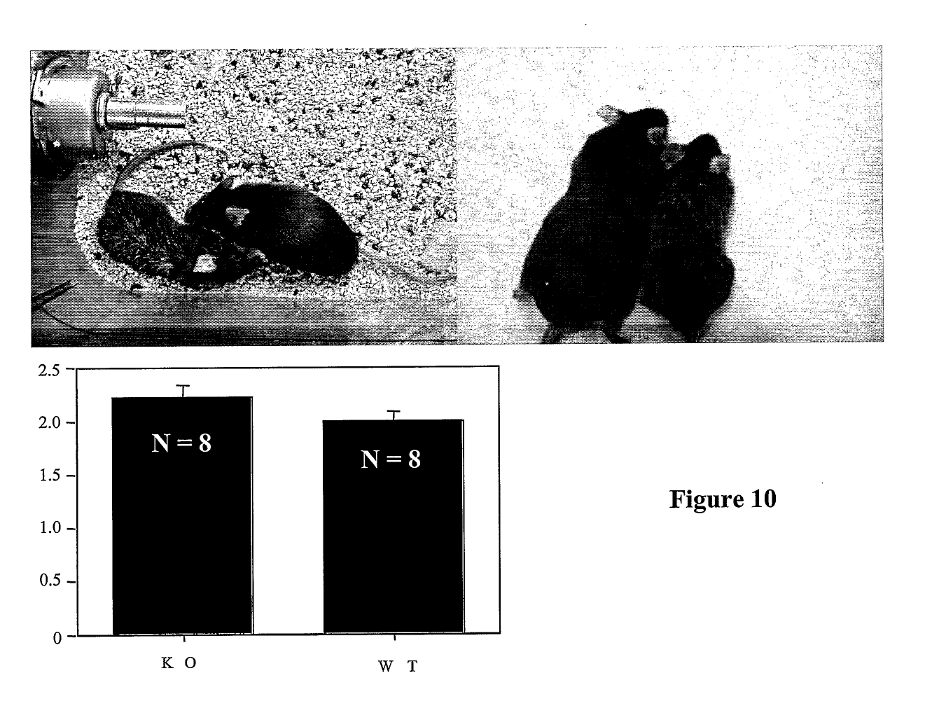

FIG. 11 shows aggregates of large mitochondria in the skeletal muscle of TR4 KO mice.

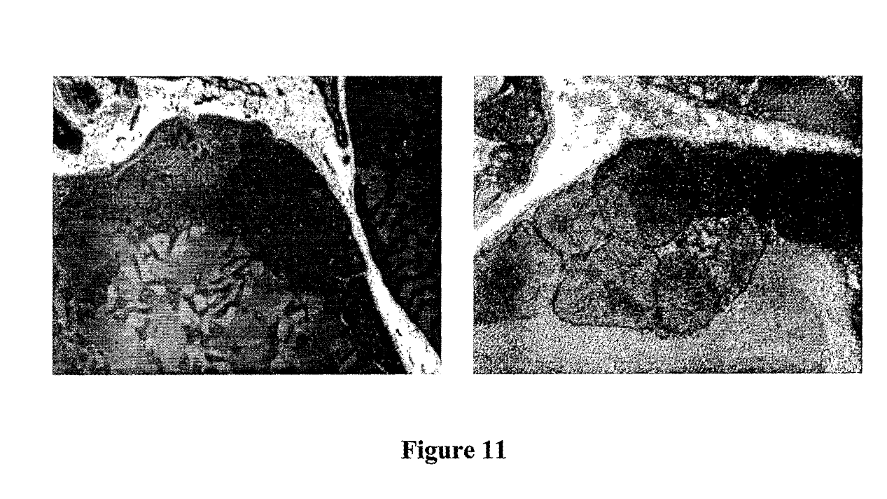

FIG. 12 shows a reduction of dermal thickness and absence of subcutaneous adipose cells were seen in 6 month old TR4^{-/-} compared with aged-matched TR4^{+/+} mice. m, muscle; f, fat; d, dermis; and e, epidermis.

FIG. 13 shows DEXA scan analyses of TR4^{-/-} mice male (n=5), and female (n=5) mice at age of 6-7 months as compared to aged/gender matched TR4^{+/+} mice. Differences in BMD of a particular gender and genotypes were analyzed by student t-test (* indicated p<0.05, and ** indicated p<0.01).

FIG. 14 shows increased adipocyte accumulation in the bone marrow of TR4 knock out mice. A sign of senile human osteoporosis is accompanied with increased adipocytes in bone marrow.

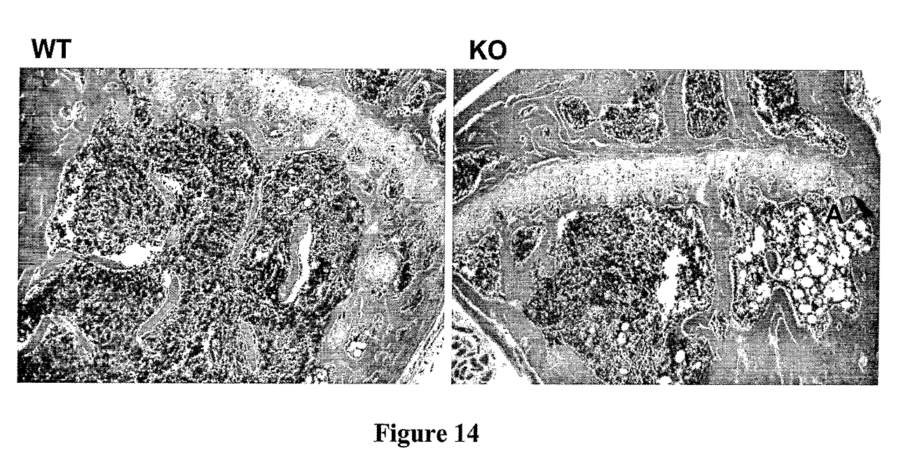

FIG. 15 shows that extramedullary haemotpoiesis is seen in the liver of six month old TR4^{-/-} mice. This is another aging phenotype.

FIG. 16 shows cell cycle profile analysis of passage 2 (P2) and P4 MEFs from TR4^{-/-} and TR4^{+/+}. TR4^{-/-} display an early G2/M arrest in P4, while TR4^{+/+} MEFs showed a normal cell cycle distribution.

FIG. 17 shows reduced ROS scavenger ability in TR4 KO mice.

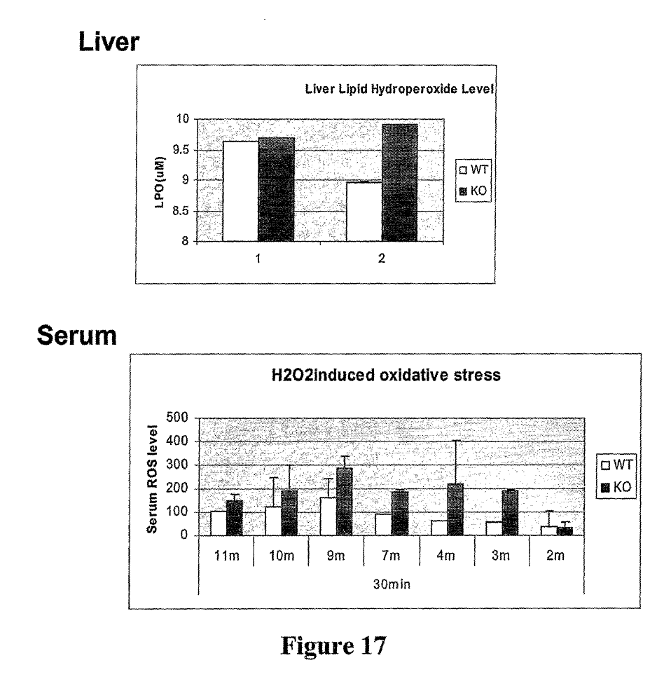

FIG. 18 shows screening for all the cell cycle, stress related, DNA repair genes. These are RNA samples from the liver.

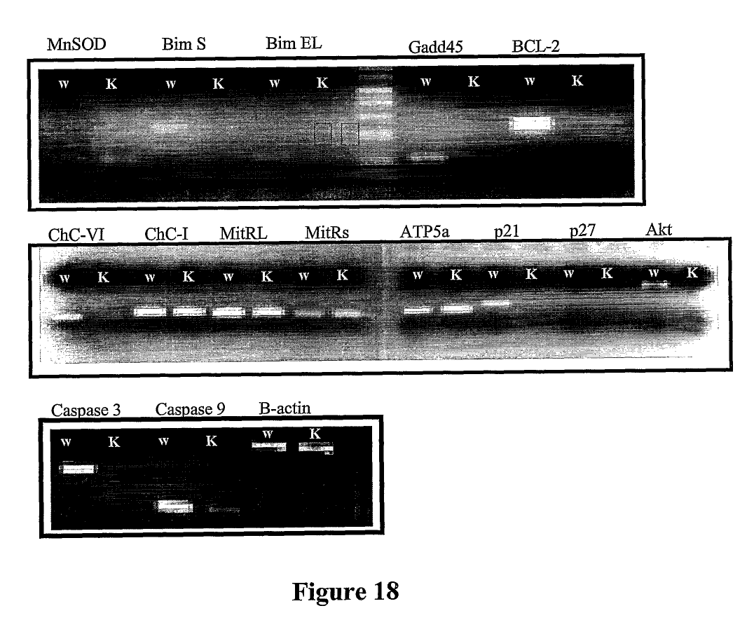

FIG. 19 shows TR4 responses to stress and regulates Gadd45α gene activity. FIG. 19A shows that H₂O₂ induces TR4 mRNA and protein expression C2C12 cells were treated with 250 μM of H₂O₂ for 2 h, then cells were harvest 0h, 4 h, 12 h, 18 r, 24 h, and 36 h post-treatment. The levels of TR4 mRNA and proteins were measured by Q-PCR and Western blotting analyses. FIG. 19B shows reduced Gadd45α expression in 6 month TR4^{-/-} muscle when compared with TR4^{+/+}. FIG. 19C shows the loss of IR-induced Gadd45α mRNA expression in TR4^{-/-} MEFs. FIG. 19D shows that several DR3-motifs as indicated in I, II, III, IV were found in Gadd45α intron 3 and exon 4. FIG. 19F shows that TR4 activates Gadd45α reporter genes (GaddLuc), which containing regions I, II, and III DR3 motif, not the reporter contain only IV DR3 regions (GaddLuc3). CV-1 cells were co-transfected with Gadd45a reporter and different amounts of TR4 (PCMX-TR4), and Luc activities were measured. FIG. 19G shows an illustration of TR4 roles in the stress-ROS-DNA damage/repair axis. FIG. 19H shows the Gadd45-Luc fusion containing VDRE.

FIG. 20 shows ROS in MED cells using flow cytometric analysis.

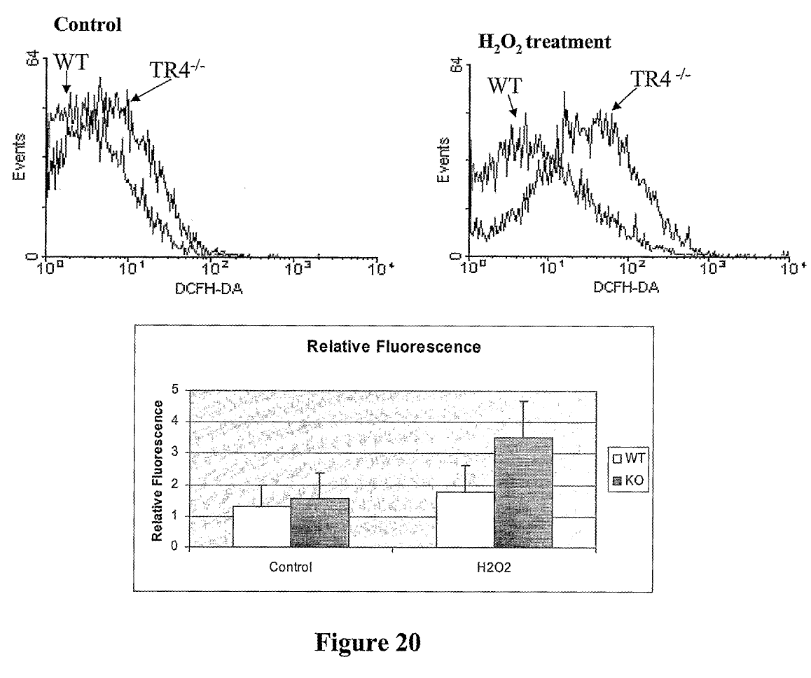

FIG. 21 shows Increased DNA single-strand breaks and cellular decay in TR4^{-/-} MEFs. FIG. 21A shows that TR4 KO MEF cells have higher SS DNA damage and ROS-induced SS DNA damage. FIG. 21B shows that TR4 overexpression rescues SS DNA damage. FIG. 21C shows that the single strand DNA breaks in, TR4^{-/-} TR4^{+/+} and TR4-transfected TR4^{-/-} were compared by DNA precipitation methods. TR4^{-/-} MEFs have more DNA breaks than TR4^{+/+} (endogenous and oxidative-stress induced), and TR4-transfected TR4^{-/-} MEFs reduced the DNA breaks.

FIG. 22 shows TR4 effects on DNA repair system. FIG. 22A shows DS DNA damage repair using an end-joint assay. FIG. 22B shows the Protective effects on pUC19 plasmid DNA break caused by hydroxyl radical produced by the Fe⁺²—H₂O₂ system. Electrophoresis was carried out a 0.8% agarose gel. Lane 1, control pUC19 DNA; lanes 2, and 5; DNA break on pUC19 by Fe⁺²—H₂O₂ treatment; lanes 3, and 4, Fe⁺²—H₂O₂ treated pUC19 in the presence of cellular proteins from TR4^{+/+} and TR4^{-/-}; and lanes 6, and 7, Fe⁺²—H₂O₂ treated pUC19 in the presence of cellular proteins from TR4^{-/-}; and TR4-transfected-TR4^{-/-} MEFs.

FIG. 23 shows the effect of H₂O₂ on TR4 KO vs. wt mice. MEFs of the indicated genotype were seeded and then harvested at indicated days to determine cell proliferation by MTT assays.

FIG. 24 shows histological analysis of cross-sections of thoracic spine of 6 month old TR4^{-/-} mice and aged matched TR4^{+/+} male mice, and reduced cortical (C, as shown in arrow) and trabecular (T, as shown in arrow) bone area.

FIG. 25 shows early onset of cellular senescence and genotoxic stress sensitivity in TR4^{-/-} MEFs. FIGS. 25A and B show hypersensitivity of TR4^{-/-} MEFs to H₂O₂ (A) and gamma irradiation (B) treatments. MEFs of the indicated genotypes were exposed to increasing doses of H₂O₂ (50-200 μM) for 2 h, and gamma irradiation (3, 6, and 9 Gys), and then cells were harvested 72 h after treatment to determine the cell amount. The percentage of cell survival was calculated by comparing with untreated cells. FIG. 25C shows the endogenous and H₂O₂-stimulated ROS were measured by flow analysis. The cellular ROS level, shown as the dichlorofluorescein fluorescence intensity in TR4^{-/-} MEFs is higher than TR4^{+/+} in both conditions. Quantification of cellular ROS levels in MEFs from TR4^{-/-}/, TR4^{+/+}, or TR4^{-/-} expression of TR4. Retrovirus containing pBabe-TR4 and pBabe vector was infected into TR4^{-/-} MEFs for 2 days and cells were treated with/without 250 μM H₂O₂, and then cellular ROS levels were determined by flow cytometric analyses.

### III. DETAILED DESCRIPTION

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods, specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Abbreviations: CAT, chloramphenicol acetyltransferase; DBD, DNA-binding domain; E2, 17β-estradiol; ER, estrogen receptor; ERE, estrogen response element; GST, glutathione S-transferase; LBD, ligand-binding domain; PR, progesterone receptor; TR2, Testicular orphan receptor 2, TR4, Testicular orphan receptor 4; RA, retinoic acid; PPARα, peroxisome proliferator-activated receptor α; CAT, chloramphenicol acetyltransferase; RAR, retinoic acid receptor; PPRE, peroxisome proliferator response element; 1,25-(OH)₂D₃, 1,25-dihydroxyvitamin D₃; Kd, equilibrium dissociation constant, TR4 associated constant; AR, androgen receptor; GR, glucocorticoid receptor; TR, thyroid hormone receptor; TR4RE, TR4 response element; TR4-N, TR4-N terminus; TR4-DL, TR4 DNA binding domain (DBD) and ligand binding domain (LBD); DR, direct repeat; HDACs, histone deacetylases; TSA, Trichostatin; EMSA, electrophoretic mobility shift assay; LUC, luciferase; -UL, minus uronolactone.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between “10” and “15.” It is also understood that each unit between two particular units are also disclosed. For example, if “10” and “15” are disclosed, then “11,” “12,” “13,” and “14” are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

A “decrease” can refer to any change that results in a smaller amount of TR4 activity. Thus, a “decrease” can refer to a reduction in an activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed.

An “increase” can refer to any change that results in a larger amount of a TR4 activity. Thus, for example, an increase in the amount in Tr4 activity can include but is not limited to a 10%, 20%, 30%, 40%, 50%, 60%, 70%, 80%, 90%, or 100% increase.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Treatment,” “treat,” or “treating” mean a method of reducing the effects of a disease or condition. Treatment can also refer to a method of reducing the disease or condition itself rather than just the symptoms. The treatment can be any reduction from native levels and can be but is not limited to the complete ablation of the disease, condition, or the symptoms of the disease or condition. Therefore, in the disclosed methods, treatment” can refer to a 10%, 20%, 30%, 40%, 50%, 60%, 70%, 80%, 90%, or 100% reduction in the severity of an established disease or the disease progression. For example, a disclosed method for reducing the effects of prostate cancer is considered to be a treatment if there is a 10% reduction in one or more symptoms of the disease in a subject with the disease when compared to native levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition.

“Obtaining a tissue sample” or “obtain a tissue sample” means to collect a sample of tissue from a subject or measure a tissue in a subject. It is understood and herein contemplated that tissue samples can be obtained by any means known in the art including invasive and non-invasive techniques. It is also understood that methods of measurement can be direct or indirect. Examples of methods of obtaining or measuring a tissue sample can include but are not limited to tissue biopsy, tissue lavage, aspiration, tissue swab, spinal tap, magnetic resonance imaging (MRI), Computed Tomography (CT) scan, Positron Emission Tomography (PET) scan, and X-ray (with and without contrast media).

Transcription activity as used herein refers to the activity a particular protein has as an activator of transcription. There are many ways that this activity can be determined, for example, CAT assays or luceriferase assays are two examples used herein.

A system refers to a collection of components which have a certain function or activity. For example, a cell that is transfected with a particular nucleic acid that is expressed can be a system that can be used for the expression of the cognate nucleic acid.

Interacts means that two (or more) molecules touch one another in a way beyond the touching that takes place because of random contacts between molecules. “Interacts” can be thought of as “binding” between two or more molecules, and therefore can have dissociation and association constants as well as equilibrium constants.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular TR4 or TR2 or AR or ER is disclosed and discussed and a number of modifications that can be made to a number of molecules including the TR4 or TR2 or AR or ER are discussed, specifically contemplated is each and every combination and permutation of TR4 or TR2 or AR or ER and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### B. COMPOSITIONS AND METHODS

The aging process is a unique feature of the life cycle of all multicellular organisms with progressive impairment, ultimate failure in homeostasis maintenance, and resultant death (Hasty, P., Campisi, J., Hoeijmakers, J., van Steeg, H., and Vijg, J. Science, 299: 1355-1359, 2003.) The accumulation of somatic damage is a main cause of the aging process. Among the various sources of somatic damage, reactive oxygen species (ROS), the natural by-products of oxidative energy metabolism in mitochondria, are considered as the ultimate cause of aging (Droge, W. Adv Exp Med Biol, 543: 191-200, 2003.)

DNA damage is a common cell death-inducing signal but the death program that is activated varies by cell type. DNases and ROS can damage DNA. The mitochondrion is a significant source of ROS that are associated with the pathogenesis of many diseases and with aging (Genova et al., Ann N Y Acad Sci, 1011: 86-100, 2004; Huang et al. Front Biosci, 9: 1100-1117, 2004.) The oxygen species that are typically linked to oxidative stress include superoxide anion, hydroxyl radical (OH), hydrogen peroxide (H₂O₂), nitric oxide (NO) and peroxynitrite (ONOO—). Although generation of these species from molecular oxygen is a normal feature of mammalian respiration, ROS directly targets DNA resulting in different lesions, such as single- or double-strand DNA breaks (Bohr, V. A., Stevnsner, T., and de Souza-Pinto, N.C. Gene, 286: 127-134, 2002). The most frequent oxidative damage to DNA is the 8-hydroxylation/oxidation of guanine base to 8-hydroxydeoxguanosine (8-OHdG). These lesions disrupt vital processes, such as transcription and replication, which can cause growth arrest or cell death. To cope with DNA damage, organisms evolved an intricate network of DNA damage repair pathways, each focusing on a different class of lesion (Lehmann, A. Curr Biol, 12: R550-551, 2002). Alterations in the genome have been considered critically important. In addition to DNA damage, ROS can cause severe damage to cellular proteins and lipids when produced at high levels by disease processes such as ischemia, atherosclerosis, diabetes, pulmonary fibrosis, neurodegenerative disorders, and arthritis.

The mitochondria theory of aging (MTA) postulates that damage to mitochondrial DNA (mtDNA) and organelles by ROS leads to loss of mitochondrial function and loss of cellular energy (Jacobs, H. T. Aging Cell, 2: 11-17, 2003). Mutations in mtDNA occur at 16 times the rate seen in nuclear DNA. Unlike nuclear DNA, mtDNA has no protective histone proteins and DNA repair is less efficient in mitochondria than in the nucleus (Mandavilli, B. S et al. Mutat Res, 509: 127-151, 2002). Free radicals leaking from mitochondria result in damage to cellular protein, lipid, and DNA throughout the cell. This damage has been implicated as a cause of aging. mtDNA deletion mutations accumulate in post-mitotic cells with age. The inefficient mitochondria survive and reproduce causing the animal to develop early onset of senescence.

Mouse models have shown that accelerated aging is a consequence of defects in genome maintenance systems. TTD mutant mice, which have deficiencies in DNA repair and gene transcription, have developed a premature aging process (de Boer et al. J. H. Science, 296: 1276-1279, 2002.)

p53 deletion mutant mice display an early onset of phenotypes associated with aging (Tyner et al. Nature, 415: 45-53, 2002) and the Ku 80 deficient mice, which have an impairment in double-strand DNA break repair system, also developed early onset of senescence (Vogel et al. Proc Natl Acad Sci USA, 96: 10770-10775, 1999; Parrinello et al. J. Nat Cell Biol, 5: 741-747, 2003.) Hence, the accelerated aging syndromes in mice with genetic defects in genome maintenance show that genome instability, driven by oxidative damage, is a primary cause of normal aging. Since defects in genome maintenance lead to accelerated aging in human and mice, it appears that normal aging is caused by inadequately repaired DNA damage. Genotype-phenotype correlations in mouse models of defects in genome maintenance can provide valuable insights into basic mechanisms of aging and natural defense systems that promote longevity. In addition to DNA repair gene mutation, deletion of klotho and SNF2-like gene PASG develops premature aging (Kuro-o et al. Nature, 390: 45-51, 1997; et al. Genes Dev, 18: 1035-1046, 2004.) The premature aging phenotype found in mice in defective mtDNA polymerase further shows the role of mitochondria in maintain the longevity. In addition to mice, several human diseases exhibit symptoms of acceleration of aging. Diseases that resemble certain aspects of accelerated aging are known as segmental progerias, because of segments of aging in each disease condition. Segmental progerias include disease of DNA-damage/repair (such as Werner's syndrome (Bohr et al. Biogerontology, 3: 89-94, 2002) and xeroderma pigmentosum), and diseases showing telomere abnormalities (such as Hutchinson-Gilford syndrome and Down's syndrome)(Brown, W. T. Curr Probl Dermatol, 17: 152-165, 1987; Martin, G. M. Natl Cancer Inst Monogr, 60: 241-247, 1982; Brown, W. T. Annu Rev Gerontol Geriatr, 10: 23-42, 1990).

The TR4 is a member of the nuclear receptor superfamily. The nuclear receptor superfamily is comprised of transcription factors that are related by sequence and structure, yet are specifically induced or repressed by a wide variety of chemical compounds. Functioning as transcription factors, nuclear receptors can control the expression of target genes and thereby direct developmental, physiological, and behavioral responses from the cellular level to that of the whole organism (Beato, M. Faseb J, 5: 2044-2051, 1991; Beato, M. and Klug, J. Hum Reprod Update, 6: 225-236, 2000). The structural features common to nuclear receptors include those required for ligand binding, dimerization, DNA binding, and transactivation. Binding of a particular receptor to a specific DNA sequence or hormone response element (HRE) within the promoter of one of its target genes is mediated by a DNA binding domain that contains two zinc finger motifs.

Both embryonic and adult tissue distribution analysis demonstrated that TR4 is expressed mainly in neural and testis during embryonic development. In situ hybridization experiments using TR4 specific probes have shown transcripts present in actively proliferating cell populations of the brain and peripheral organs during embryonic development. The expression of TR4 at sites of sensory innervation and in sensory organs throughout embryogenesis indicate an important role for these receptors in this critical aspect of nervous system development. Additionally, high expression of TR4 in the developing brain and spinal cord, including specific expression in motor neurons, show that these receptors can be involved in the proper development of movement and limb coordination (Young et al. J Biol Chem, 272: 3109-3116, 1997).

TR4 is closely related to the retinoic X receptor (RXR), and binds to AGGTCA DNA sequence motifs in direct repeat orientation, with variable spacing, in the promoters of its target genes (Chang et al. Proc Natl Acad Sci USA, 91: 6040-6044, 1994). Therefore, TR4 can directly influence gene activation by directly binding to DNA and activating genes such as ApoE and Vitamin D receptor (VDRE) (Kim et al. J Biol Chem, 278: 46919-46926, 2003; Lee et al. J Biol Chem, 274: 16198-16205, 1999). On the other hand, TR4 acts as a suppressor to influence other receptor functions, such as RXR/retinoic acid receptor (RAR), androgen receptor (AR), and estrogen receptor (ER) (Lee et al. J Biol Chem, 273: 13437-13443, 1998; Lee et al Proc Natl Acad Sci USA, 96: 14724-14729, 1999; Shyr et al. J Biol Chem, 277: 14622-14628, 2002) by competition for the same DNA binding sites or through protein-protein interactions.

In vitro data show that TR4 functions as a master regulator to modulate many signaling pathways. To investigate TR4 function, mice lacking TR4 (TR4 KO) via targeted gene disruption have been created (Collins et al. Proc Natl Acad Sci USA, 101:15058-15063, 2004, herein incorporated by reference in its entirety for its teaching concerning TR4 KO mice). The lambda KOS system was used to derive a TR4 targeting vector, and three independent genomic clones spanning exons 4-10 were isolated. The targeting vector was derived from one clone and contained a 2173 bp deletion that included most of exon 4 and all of exon 5. The genomic sequence encoding the DBD of TR4 was replaced by a Lac-Z/Neo selection cassette. The Not I linearized vector was electroporated into strain 129SvEvbrd (LEX1) embryonic stem (ES) cells, and G418/FIAU-resistant ES cell clones were isolated and screened by Southern blot for homologous recombination of the mutant DNA. One targeted ES cell clone was injected into blastocysts of strain C57BL/6 (albino), which were then inserted into pseudopregnant female mice for continuation of fetal development. Resulting chimeric male mice were then mated to C57BL/6 (albino) females to generate animals heterozygous for the mutation. The TR4 KO mice demonstrate high rates of early postnatal mortality, as well as significant growth retardation. TR4 KO mice also display reproductive defects, in which reduced fertility was seen in both genders (Mu et al. Mol Cell Biol, 24: 5887-5899, 2004).

The surviving adult TR4 KO mice develop growth impairments, including growth retardation, hypoglycemia, and mild late-onset myopathy where mitochondria-like proliferation inclusions were found. Furthermore, decline of mitochondria function is often linked to aging related syndrome (Roubertoux et al. Nat Genet, 35: 65-69, 2003). By 6 months, most of the mice develop kyphosis and a sign of osteoporosis with a reduced bone mineral density (BMD). A premature ovarian failure was observed in three 6 month-old TR4 KO females, in which there was no active estrus cycle and complete anovulation. All those phenotypes indicate TR4 KO mice develop premature aging. TR4 KO mice embryonic fibroblast (MEF) cells display a dramatic reduction in replicative lifespan. Emerging late age-onset phenotypes observed in TR4 KO mice, abnormal mitochondria proliferation, and reduction of MEF replicative lifespan show that TR4 plays an important role in maintaining the genome stability, and loss of TR4 in mice can lead to development of systemic problems which cause the premature aging process.

As discussed above, TR4 KO mice, in general, have shorter life spans, and most of the mice won't live over one year. TR4 KO mice also have high pre-puberty mortality with a 35% mortality rate. The surviving adult KO mice develop growth impairments, including growth retardation, hypoglycemia, and mild late-onset myopathy where mitochondria-like proliferation inclusions were found. Decline of mitochondria function is often linked to aging related syndrome (Martin et al. Nature, 429: 417-423, 2004; Stevnsner et al. Exp Gerontol, 37: 1189-1196, 2002.) By 6 months, most of the mice develop kyphosis and a sign of osteoporosis with a reduced bone mineral density (BMD). A premature ovarian failure was observed in three 6 month-old TR4 KO females, in which there was no active estrus cycle and complete anovulation. All above phenotypes indicate TR4 KO mice develop premature aging. TR4 KO mouse embryonic fibroblast (MEF) cells display a dramatic reduction in replicative lifespan. Emerging late age-onset phenotypes observed in TR4 KO mice, abnormal mitochondria proliferation, and reduction of MEF replicative lifespan shows that TR4 plays an important role in maintaining the genome stability and loss of TR4 in mice that can lead to development of systemic problems that cause premature aging.

TR4 KO mice developed an early onset of aging progression, which provides a model to study the initiation and progression of the aging process through monitoring the changes of multiple organ systems throughout the life span. Determination of the stages, as well as gender differences, and organs which are targeted by aging is necessary to dissect the mechanisms that are associated with the premature aging process in TR4 KO mice. Most importantly, the changes between earlier stage vs. later stages in particular organs/systems during this aging process can be used to identify factors operating in early or mid-life origins and consequences that occur in late stage, all of which are essential for understanding the aging process. Many organs and systems can be examined. Examples include skin, muscle, bone, cardiovascular function, urinary function, reproductive systems, and immune systems through all segments of the life span, from neonatal (P7), before puberty (1 month), young adulthood (2-3 month), mid-age (4-6 month), mid-late (7 month to 1 yr), to late-life (over 1 year).

As discussed above, progressive decline in mitochondria function accompanies aging. One of the theories of mitochondria aging (MTA) is that reactive oxidative species (ROS), natural by-products of oxidative energy metabolism in mitochondria, which directly target DNA result in different lesions (Ames et al. J Alzheimers Dis, 6: 117-121, 2004; Liu et al. Ann N Y Acad Sci, 959: 133-166, 2002; Ames et al. Ann N Y Acad Sci, 1019: 406-411, 2004). The burden of ROS is largely counteracted by antioxidant defense and DNA repair systems, with inadequately repaired DNA damage eventually leading to aging. In young organisms, there are a large number of small mitochondria that provide needed ATP, however there are many large mitochondria in aged organisms. These larger mitochondria are not as bio-energetically efficient as the youthful, normal, small mitochondria (Bertoni-Freddari et al. Ann N Y Acad Sci, 717: 137-149, 1994; Miquel, J. Exp Gerontol, 33: 113-126, 1998; Lee et al. J Steroid Biochem Mol Biol, 81: 291-308, 2002.) Electron microscopy examination of skeletal muscle from 6 month old TR4 KO showed enlarged and abnormal proliferation mitochondria, an indication of mitochondrial functional decline. TR4 KO mice that have mitochondrial dysfunction generated excess ROS burden to induce DNA damage. Furthermore, the impairment of DNA repair capacity eventually results in accelerated aging in TR4 KO mice. Mitochondria function, mitochondrial DNA integrity, ROS status, and DNA damage can be examined in different stages of TR4 KO mice, for comparisons with their wild type littermates.

MEF rapid senesce is the result of severe oxidative stress which induces extensive DNA damage and/or chromosomal aberrations and is a landmark of aging cells (Davis et al. J Cell Sci, 116: 1349-1357, 2003). TR4 KO MEF cells display a rapid senescence, at which TR4 KO MEF cells arrest at G2/M phase after four population doublings (P4), indicating that TR4 KO MEF cells fail to overcome replicative senescence that is caused by oxidative stress. MEF cells derived from TR4 KO and wild type mice can be examined to determine the mechanisms underlying the replicative senescence and determine its contribution to accelerated aging in mice. MEF cells are challenged with DNA-damage inducers, such as hydrogen peroxide (H₂O₂) and UV, and then ROS status, the degree of DNA damage, DNA repair ability, DNA replication, and cell survival can be measured and compared. Viral TR4 infection is used to rescue the defects in TR4 KO MEF to confirm the roles of TR4. The known genes related to the stress-response, cell survival, and DNA damage/repair systems are compared between TR4 KO and wt MEF cells. In addition, microarray analysis can be used to identify the TR4 targeted genes, which are responsible for the TR4 KO MEF rapid replicative senescence.

TR4 is the first steroid nuclear receptor shown to have a biological function closely linked to aging. Therefore, identification of signaling pathways that modulate TR4 activity are important for understanding the fundamental actions of TR4, and how this relates to the aging process. Generally, steroid hormone receptor activity can be altered by either agonists (or ligands) or by modulating the expression of receptor to change the receptor sensitivity. Aging-related hormones, like dehydroepiandrosterone (DHEA), DHEAS, thyroid, sex hormones, and cortisol can modulate TR4 activity. TR4 targeted genes and TR4 functions (its roles in DNA damage, repair, and cell proliferation response to H₂O₂) can be examined by using the MEF cell system. The modulation of TR4 activity can also be achieved via regulation of TR4 expression level, therefore the 5′-TR4 can reveal how environment factors such as stress influence TR4 activity. It has been found that stresses like starvation and hypoxia can induce TR4 expression, which shows that TR4 is a stress-sensor and is able to respond to environmental threats and protect individuals from damage. Therefore, a 5′-TR4-promoter Luc-reporter system as well as MEF cells from TR4 KO and wild-type mice can be used to show how those potential environment factors and aging related hormone modulate TR4 activity. A 6 kb of TR45′-flanking region and its serial deletions have been cloned and constructed into Luciferase reporter genes.

TR4 activity is shown herein to have an effect on aging. Specifically, it is disclosed herein, that a decrease in TR4 activity results in increased signs of aging, such as mitochondrial DNA damage, inadequate DNA repair systems, and severe oxidative stress. Thus, decreased TR4 activity can play a prime role in the development of premature aging as well as the common diseases and disorders associated with aging in older subjects. Therefore, disclosed are methods of generating a model for premature aging comprising generating a TR4 knock out animal and assaying the animal for characteristics of aging. Also disclosed herein are methods of testing a subject for premature aging comprising performing an assay for premature aging, wherein the subject has a TR4 deficiency.

Furthermore, the techniques and methods disclosed herein can be used to assess the likelihood a subject will develop a condition due to decreased TR4 activity. Thus, disclosed are methods for diagnosing the likelihood of a subject to develop premature aging comprising taking a tissue sample from the subject and assaying for TR4 activity, wherein a decrease in TR4 activity indicates premature aging. It is understood that subjects with decreased TR4 activity can have increased signs of aging. Therefore, it is understood and herein contemplated that a subject with decreased TR4 activity will likely develop premature aging. Similarly, the disclosed methods can be used to diagnose the likelihood a subject will develop signs of aging. Thus also disclosed are methods for diagnosing the likelihood of a subject to develop premature signs of aging comprising taking a tissue sample from the subject and assaying for TR4 activity, wherein a decrease in TR4 activity indicates aging. It is understood and herein contemplated that subjects with decreased TR4 activity can have increased DNA damage, including mitochondrial DNA, and any of the other signs or symptoms associated with aging that are known in the art. It is also understood that a subject can be a cell, mammal, mouse, rat, pig, dog, cat, cow, horse, monkey, chimpanzee or other none human primate, or human.

The disclosed methods can also be used to diagnose a condition. Thus disclosed are methods of diagnosing a subject with premature aging comprising a) obtaining a tissue sample, and b) assaying for TR4 activity, wherein a lack of TR4 activity indicates premature aging. Also disclosed is a method of diagnosing a subject with premature aging comprising a) obtaining a tissue sample, and b) assaying for TR4 activity, wherein a lack of or reduced TR4 activity indicates premature aging. It is understood that various tissue samples can be used with the disclosed methods. Specifically disclosed are methods, wherein the tissue sample is blood, muscle, bone, kidney, or liver tissue.

Due to the effects of TR4 activity on principal activities associated with aging (e.g., those associated with ROS such as ischemia, atherosclerosis, diabetes, pulmonary fibrosis, neurodegenerative disorders, and arthritis), agents that can increase TR4 activity can be used to treat these conditions. Thus, disclosed are methods for screening drugs for an effect on aging comprising administering the drug to an animal and assaying for TR4 activity, wherein an increase in TR4 activity indicates a drug that can be used to treat aging. Also disclosed are methods for screening drugs for an effect on aging comprising administering the drug to a TR4 deficient mouse and assaying for TR4 activity, wherein an increase in TR4 activity indicates a drug that can treat aging.

Also disclosed are methods of treating a subject with signs of aging comprising administering to the subject an agent that modulates TR4 activity, wherein an increase in TR4 activity reduces aging. Also disclosed are methods of treating a subject with premature aging comprising administering to the subject an agent that modulates TR4 activity, wherein an increase in TR4 activity reduces premature aging.

An example of a ligand of TR4 is DHEA and its derivatives. Dehydroepiandrosterone (DHEA), in its sulphated form (DHEA-S), is the most plentiful adrenal steroid circulating in the human bloodstream. Along with the major human glucocorticoid, cortisol, DHEA is produced in the adrenal cortex. DHEA circulates primarily as DHEA-S, which is generally metabolically inactive. As cells take DHEA-S from the blood, they reconvert it into DHEA, and possibly other metabolites. The pregnenolone metabolite, 17-hydroxypregnolone, is the common parent molecule for both DHEA and cortisol. Humans, along with some other primates, are unique in having adrenals which secrete large amounts of DHEA/DHEA-S.

Disclosed are methods for testing a compound for an effect on aging comprising administering the compound to an animal and assaying for TR4 activity, wherein an increase in TR4 activity indicates a compound that can be used to treat the effects of aging. It is understood that testing, screening, or evaluation of compositions (e.g., a compound or drug) for effects on aging can utilize TR4 knockout animals. Therefore, also disclosed are methods of testing a composition for an effect on aging comprising administering the composition to a TR4 knockout animal, and performing an assay related to aging, wherein a change in the assay relative to a control indicates the composition has an effect on aging.

Disclosed are methods for evaluating whether a treatment with a compound should be performed due to the effect the treatment has on aging, wherein the compound modulates the TR4 activity, the method comprising a) exposing cells to the compound, and

b) evaluating TR4 activity in the presence of the compound, wherein a change in the TR4 activity of the subject, relative to the TR4 activity of a subject that has not been exposed to the compound, indicates that the compound modulates TR4 activity, and wherein a decrease in TR4 activity indicates a negative effect on aging, providing an indication that treatment with the compound may not be indicated.

The methods and compositions disclosed herein are useful in treating aging and premature aging. One aspect of premature aging involves the Hutchinson-Gilford progeria syndrome (HGPS), commonly referred to as progeria. The landmarks of aging include DNA damage and chromosomal aberrations. Evidence exists for the decline in DNA repair and the accumulation of DNA damage in several different types of cells taken from elderly subjects. Elderly patients' blood and skin cells have less capacity to repair themselves than those from young adults. Furthermore, aging white blood cells with their higher level of DNA damage can explain some of the decline in immune function associated with aging.

### C. COMPOSITIONS

### 1. Molecules that Inhibit TR2/TR4 Interactions

a) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of TR4 or the genomic DNA of TR4 or they can interact with the polypeptide TR4. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RnaseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d})less than 10⁻⁶. It is more preferred that antisense molecules bind with a k_{d} less than 10⁻⁸. It is also more preferred that the antisense molecules bind the target molecule with a k_{d} less than 10⁻¹⁰. It is also preferred that the antisense molecules bind the target molecule with a k_{d} less than 10⁻¹².

A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶. It is more preferred that the aptamers bind the target molecule with a k_{d} less than 10⁻⁸. It is also more preferred that the aptamers bind the target molecule with a k_{d} less than 10⁻¹⁰. It is also preferred that the aptamers bind the target molecule with a k_{d} less than 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10 fold lower than the k_{d} with a background binding molecule. It is more preferred that the aptamer have a k_{d} with the target molecule at least 100 fold lower than the k_{d} with a background binding molecule. It is more preferred that the aptamer have a k_{d} with the target molecule at least 1000 fold lower than the k_{d} with a background binding molecule. It is preferred that the aptamer have a k_{d} with the target molecule at least 10000 fold lower than the k_{d} with a background binding molecule. It is preferred when doing the comparison for a polypeptide for example, that the background molecule be a different polypeptide. For example, when determining the specificity of TR2, TR4, AR, or ER, or fragments thereof, aptamers, the background protein could be serum albumin. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intennolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶. It is more preferred that the triplex forming molecules bind with a k_{d} less than 10⁻⁸. It is also more preferred that the triplex forming molecules bind the target molecule with a k_{d} less than 10⁻¹⁰. It is also preferred that the triplex forming molecules bind the target molecule with a k_{d} less than 10⁻¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukaryotic cells. (Yuan et al., Proc. Natl. Acad. Sci. USA 89:8006-8010 (1992); WO 93/22434 by Yale; WO 95/24489 by Yale; Yuan and Altman, EMBO J. 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162

b) Antibodies

(1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with TR4 or fragments thereof such that TR4 are inhibited from performing transactivation activity. Antibody also includes, chimeric antibodies and hybrid antibodies, with dual or multiple antigen or epitope specificities, and fragments, such as F(ab′)2, Fab′, Fab and the like, including hybrid fragments, as well as conjugates of antibody fragments and antigen binding proteins (single chain antibodies) as described, for example, in U.S. Pat. No. 4,704,692, the contents of which are hereby incorporated by reference. Antibodies that bind the disclosed regions of TR4 or fragments thereof, such that TR4 decrease their transactivation activity are also disclosed. The antibodies can be tested for their desired activity using the in vitro assays described herein, or by analogous methods, after which their in vivo therapeutic and/or prophylactic activities are tested according to known clinical testing methods. Thus, fragments of the antibodies that retain the ability to bind their specific antigens are provided. Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

The term “monoclonal antibody” as used herein refers to an antibody obtained from a substantially homogeneous population of antibodies, i.e., the individual antibodies within the population are identical except for possible naturally occurring mutations that can be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces mono clonal antibodies. For example, monoclonal antibodies of the invention can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes can be immunized in vitro, e.g., using the binding domains of the compositions described, herein, such as the ligand binding domain, described herein.

The monoclonal antibodies can also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment can be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods of the invention serves to lessen the chance that an antibody administered to a human will evoke an undesirable immune response.

(2) Human Antibodies

The human antibodies of the invention can be prepared using any technique. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1):86-95, 1991). Human antibodies of the invention (and fragments thereof) can also be produced using phage display libraries (Hoogenboom et al., J. Mol. Biol., 227:381, 1991; Marks et al., J. Mol. Biol., 222:581, 1991).

The human antibodies of the invention can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

(3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a nonhuman (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies can also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

(4) Administration of Antibodies

Administration of the antibodies can be done as disclosed herein. Nucleic acid approaches for antibody delivery also exist. The broadly neutralizing antibodies and antibody fragments of the invention can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

c) Compositions Identified by Screening with Disclosed Compositions/Combinatorial Chemistry

(1) Combinatorial Chemistry

The disclosed compositions can be used as targets for any combinatorial technique to identify molecules or macromolecular molecules that interact with the disclosed compositions in a desired way. The nucleic acids, peptides, and related molecules disclosed herein, such as TR4 or fragments thereof, can be used as targets for the combinatorial approaches. Also disclosed are the compositions that are identified through combinatorial techniques or screening techniques in which the compositions disclosed in herein, such as TR4 or fragments thereof, or portions thereof, are used as the target in a combinatorial or screening protocol.

It is understood that when using the disclosed compositions in combinatorial techniques or screening methods, molecules, such as macromolecular molecules, will be identified that have particular desired properties such as inhibition or stimulation or the target molecule's function. The molecules identified and isolated when using the disclosed compositions, such as, TR4 fragments thereof, are also disclosed. Thus, the products produced using the combinatorial or screening approaches that involve the disclosed compositions, such as, TR4 or fragments thereof, are also considered herein disclosed.

Combinatorial chemistry includes but is not limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which either have de novo activity or a modified activity. For example, phage display libraries have been used to isolate numerous peptides that interact with a specific target. (See for example, U.S. Pat. No. 6,031,071; 5,824,520; 5,596,079; and 5,565,332 which are herein incorporated by reference at least for their material related to phage display and methods related to combinatorial chemistry)

A preferred method for isolating proteins that have a given function is described by Roberts and Szostak (Roberts R. W. and Szostak J. W. Proc. Natl. Acad. Sci. USA, 94(23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA, to be translated. In addition, because of the attachment of the puromycin, a peptidyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997)).

Another preferred method for combinatorial methods designed to isolate peptides is described in Cohen et al. (Cohen B. A., et al., Proc. Natl. Acad. Sci. USA 95(24):14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that interactions between synthetic or engineered peptide sequences could be identified which bind a molecule of choice. The benefit of this type of technology is that the selection is done in an intracellular environment. The method utilizes a library of peptide molecules that are attached to an acidic activation domain. A peptide of choice, for example a portion of TR2, TR4, AR, or ER, is attached to a DNA binding domain of a transcriptional activation protein, such as Gal 4. By performing the two-hybrid technique on this type of system, molecules that bind the portion of TR2, TR4, AR, or ER, can be identified.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768 and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514).

Screening molecules similar to TR4 or fragments thereof for inhibition or activation of TR4 activity is a method of isolating desired compounds.

As used herein combinatorial methods and libraries included traditional screening methods and libraries as well as methods and libraries used in iterative processes.

(2) Computer Assisted Drug Design

The disclosed compositions can be used as targets for any molecular modeling technique to identify either the structure of the disclosed compositions or to identify potential or actual molecules, such as small molecules, which interact in a desired way with the disclosed compositions. The nucleic acids, peptides, and related molecules disclosed herein can be used as targets in any molecular modeling program or approach.

It is understood that when using the disclosed compositions in modeling techniques, molecules, such as macromolecular molecules, will be identified that have particular desired properties such as inhibition or stimulation or the target molecule's function. The molecules identified and isolated when using the disclosed compositions, such as TR4, and/or fragments thereof, are also disclosed. Thus, the products produced using the molecular modeling approaches that involve the disclosed compositions, such as TR4 and/or fragments thereof, are also considered herein disclosed.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling, and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, QSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

d) Methods of Identifying Activators of TR4

Disclosed are methods of identifying an activator of TR4, comprising incubating a library of molecules with TR4 forming a mixture, and identifying the molecules that activate TR4, wherein the activity comprises an upregulation of TR4.

Also disclosed are compositions produced by any of the processes as disclosed herein, as well as compositions capable of being identified by the processes disclosed herein.

Disclosed are methods of manufacturing a composition for enhancing the interaction between TR4 and a ligand thereof, comprising synthesizing the enhancers as disclosed herein.

Also disclosed are methods that include mixing a pharmaceutical carrier with the activator of TR4 as disclosed herein, and produced by any of the disclosed methods.

Disclosed are methods of identifying activators of TR4 comprising, a) administering a composition to a system, wherein the system supports TR4 activity, b) assaying the effect of the composition on the amount of TR4 in the system, and c) selecting a composition which causes a decrease in the amount of TR4 present in the system relative to the system without the addition of the composition.

Also disclosed are methods of identifying activators of TR4 transcription activity comprising, a) administering a composition to a system, wherein the system supports TR4 transcription activity, b) assaying the effect of the composition on the amount of TR4 transcription activity in the system, and c) selecting a composition which causes an increase in the amount of TR4 transcription activity present in the system relative to the system without the addition of the composition.

### 2. Aspects Applicable to all Compositions

a) Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods can differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

b) Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization can involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions can provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

c) Nucleic Acids

There are a variety of molecules disclosed herein that are nucleic acid based, including for example the nucleic acids that encode, for example TR4 and/or fragments thereof, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

(1) Nucleotides and Related Molecules

A nucleotide is a molecule that contains a base moiety, a sugar moiety, and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-amino adenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

(2) Sequences

There are a variety of sequences related to the genes of AR, ER, TR2, TR4, and/or fragments, which can be found at Genbank, at for example, http://www.pubmed.gov and these sequences and others are herein incorporated by reference in their entireties as well as for individual subsequences contained therein.

The disclosed sequences and variants can be founding Genbank. It is understood that the description related to this sequence is applicable to any sequence unless specifically indicated otherwise. Those of skill in the art understand how to resolve sequence discrepancies and differences and to adjust the compositions and methods relating to a particular sequence to other related sequences. Primers and/or probes can be designed for any sequence given the information disclosed herein and known in the art.

(3) Primers and Probes

Disclosed are compositions including primers and probes, which are capable of interacting with the AR, ER, TR2, or TR4 nucleic acids as disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the TR4 and/or fragments thereof, nucleic acid or region of the TR4 and/or fragments thereof, nucleic acid or they hybridize with the complement of the TR4 and/or fragments thereof nucleic acid or complement of a region of the TR4 and/or fragments thereof nucleic acid.

d) Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

(1) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as nucleic acids encoding TR4 and/or fragments thereof into the cell without degradation and include a promoter yielding expression of the gene in the cells into which it is delivered. In some embodiments the vectors are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone, as well as lentiviruses. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trains.

(a) Retroviral Vectors

A retrovirus is a virus belonging to the virus family of Retroviridae, including any types, subfamilies, genus, or tropisms. Retroviral vectors, in general, are described by Verma, I M, Retroviral vectors for gene transfer. In Microbiology-1985, American Society for Microbiology, p. 229-32, Washington, (1985), which is incorporated by reference herein. Examples of methods using retroviral vectors for gene therapy are described in U.S. Pat. Nos. 4,868,116 and 4,980,286; PCT applications WO 90/02806 and WO 89/07136; and Mulligan (1993) Science 260:926-32; the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(b) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993);

Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virions are generated in a cell line such as the human 293 cell line. In another preferred embodiment both the E1 and E3 genes are removed from the adenovirus genome.

(c) Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The vectors of the present invention thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and can contain upstream elements and response elements.

(d) Large Payload Viral Vectors

Molecular genetic experiments with large human herpes viruses have provided a means whereby large heterologous DNA fragments can be cloned, propagated and established in cells permissive for infection with herpes viruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

Other useful systems include, for example, replicating and host-restricted non-replicating vaccinia virus vectors.

(2) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed compositions or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci. USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials can be in solution, suspension (for example, incorporated into microparticles, liposomes, or cells). These can be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunol. Rev., 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochim. et Biophys. Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

(3) In Vivo/Ex Vivo

As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

e) Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and can contain upstream elements and response elements.

(1) Viral Promoters and Enhancers

Preferred promoters controlling transcription from vectors in mammalian host cells can be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell. Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell. Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer can be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) can also contain sequences necessary for the termination of transcription which can affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

(2) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker can be a selectable marker. Examples of suitable selectable markers for mammalian cells are dihydrofolate reductase (DHFR), thymidine kinase, neomycin, neomycin analog G418, hygromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

f) Peptides

(1) Protein Variants

As discussed herein there are numerous variants of the TR4 proteins and/or fragments thereof that are known and herein contemplated. In addition, to the known functional TR4 and/or fragments thereof species homologs there are derivatives of the TR4 proteins and/or fragments thereof, which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof can be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| alanine | AlaA |
| allosoleucine | AIle |
| arginine | ArgR |
| asparagine | AsnN |
| aspartic acid | AspD |
| cysteine | CysC |
| glutamic acid | GluE |
| glutamine | GlnK |
| glycine | GlyG |
| histidine | HisH |
| isolelucine | IleI |
| leucine | LeuL |
| lysine | LysK |
| phenylalanine | PheF |
| proline | ProP |
| pyroglutamic acid | Glu |
| serine | SerS |
| threonine | ThrT |
| tyrosine | TyrY |
| tryptophan | TrpW |
| valine | ValV |

TABLE 2
Amino Acid Substitutions
Original Residue Exemplary Conservative Substitutions,
others are known in the art.

| | |
|---|---|
| Ala | ser |
| Arg | lys, gln |
| Asn | gln; his |
| Asp | glu |
| Cys | ser |
| Gln | asn, lys |
| Glu | asp |
| Gly | pro |
| His | asn; gln |
| Ile | leu, val |
| Leu | ile; val |
| Lys | arg; gln; |
| Met | Leu; ile |
| Phe | met; leu; tyr |
| Ser | thr |
| Thr | ser |
| Trp | tyr |
| Tyr | trp; phe |
| Val | ile; leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also can be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence can not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that protein in the particular organism from which that protein arises is also known and herein disclosed and described.

g) Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material can be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions can be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials can be in solution, suspension (for example, incorporated into microparticles, liposomes, or cells). These can be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

(1) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers can be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH1. The compositions can be administered intramuscularly or subcutaneously. Other compounds will be administered according to standard procedures used by those skilled in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

The pharmaceutical composition can be administered in a number of ways depending on whether local or systemic treatment is desired, and on the area to be treated. Administration can be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like can be necessary or desirable.

Compositions for oral administration include powders or granules, suspensions or solutions in water or non-aqueous media, capsules, sachets, or tablets. Thickeners, flavorings, diluents, emulsifiers, dispersing aids or binders can be desirable.

Some of the compositions can potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

(2) Therapeutic Uses

Effective dosages and schedules for administering the compositions can be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N. J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

Following administration of a disclosed composition, such as an antibody or other molecule, such as fragment of TR4, for forming or mimicking a TR4/ligand, for example, the efficacy of the therapeutic antibody or fragment can be assessed in various ways well known to the skilled practitioner. For instance, one of ordinary skill in the art will understand that a composition, such as an antibody or fragment, disclosed herein is efficacious in forming or mimicking a TR4 interaction in a subject by observing, for example, that the composition reduces the amount of TR4 activity. The TR4 activity can be measured using assays as disclosed herein. Any change in activity is disclosed, but a 5%, 10%, 15%, 20%, 25%, 30%, 35%, 40%, 45%, 50%, 55%, 60%, 65%, 70%, 75%, 80%, 90%, or a 95% reduction in AR or ER activity are also disclosed.

Other molecules that interact with TR4 which do not have a specific pharmaceutical function, but which can be used for tracking changes within cellular chromosomes or for the delivery of diagnostic tools for example can be delivered in ways similar to those described for the pharmaceutical products.

The disclosed compositions and methods can also be used for example as tools to isolate and test new drug candidates for a variety of TR4 related diseases such as those associated with aging and premature aging.

h) Chips and Micro Arrays

Disclosed are chips where at least one address is the sequences or part of the sequences set forth in any of the nucleic acid sequences disclosed herein. Also disclosed are chips where at least one address is the sequences or portion of sequences set forth in any of the peptide sequences disclosed herein.

Also disclosed are chips where at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.

i) Computer Readable Mediums

It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

Disclosed are computer readable mediums comprising the sequences and information regarding the sequences set forth herein.

### 3. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended.

### D. METHODS OF MAKING THE COMPOSITIONS

The compositions disclosed herein and the compositions necessary to perform the disclosed methods can be made using any method known to those of skill in the art for that particular reagent or compound unless otherwise specifically noted.

### 1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug Chem. 5:3-7 (1994).

### 2. Peptide Synthesis

One method of producing the disclosed proteins is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant GA (1992) Synthetic Peptides: A User Guide. W. H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides can be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

### 3. Process for Making the Compositions

Disclosed are processes for making the compositions as well as making the intermediates leading to the compositions. There are a variety of methods that can be used for making these compositions, such as synthetic chemical methods and standard molecular biology methods. It is understood that the methods of making these and the other disclosed compositions are specifically disclosed.

Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate including a human, ape, monkey, orangutan, or chimpanzee.

Also disclosed are animals produced by the process of adding to the animal any of the cells disclosed herein.

### E. METHODS OF USING THE COMPOSITIONS

### 1. Methods of Using the Compositions as Research Tools

The compositions can be used for example as targets in combinatorial chemistry protocols or other screening protocols to isolate molecules that possess desired functional properties related to TR4. For example, TR4 and its interaction domains can be used in procedures that will allow the isolation of molecules or small molecules that mimic their binding properties. Libraries of molecules can be screened for interaction with TR4 by incubating the potential binding molecules with TR4 and then isolating those that are specifically active. There are many variations to this general protocol.

The disclosed compositions can also be used diagnostic tools related to diseases such as those associated with aging.

The disclosed compositions can be used as discussed herein as either reagents in micro arrays or as reagents to probe or analyze existing microarrays. The disclosed compositions can be used in any known method for isolating or identifying single nucleotide polymorphisms. The compositions can also be used in any known method of screening assays, related to chip/micro arrays. The compositions can also be used in any known way of using the computer readable embodiments of the disclosed compositions, for example, to study relatedness or to perform molecular modeling analysis related to the disclosed compositions.

### F. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Aging Appearance in 6 Month Old Tr4 KO Mice

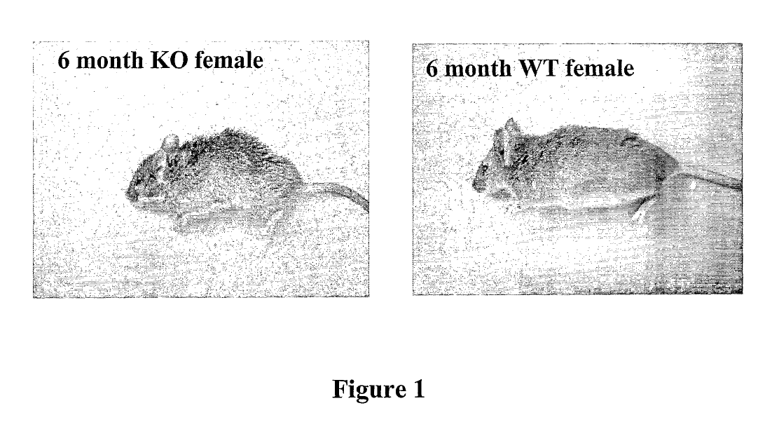

TR4 KO mice, in general, have a shorter life span and have high pre-puberty mortality with a 35% mortality rate. The surviving adult KO mice develop growth impairments, including growth retardation, hypoglycemia, and aging phenotype. As shown in FIG. 1, TR4 KO mice have greasy skin, hair loss, drooped eye lids, and long nails, as compared with wt littermates.

a) Abnormal Enlargement of Mitochondria Accumulation in Ko Mice Skeletal Muscle.

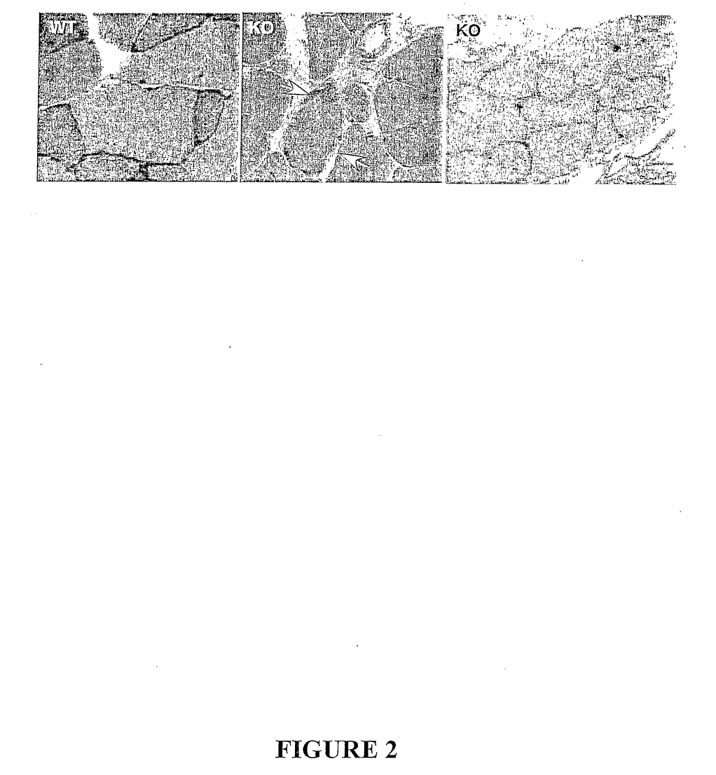

As mice grow, TR4 KO adult mice significantly decreased performance in neuromuscular coordination and displayed very poor muscle strength during movement. Most TR4 KO mice dragged their hind limbs by the age of 4-5 months, and some TR4 KO mice even have difficulty moving their hind limbs. Pathological examinations on 6-9 months-old mice muscle found muscle atrophy, small and irregular muscle fibers, a few degenerated fibers, and a large number of inclusions indicating an abnormal mitochondria proliferation (FIG. 2A), and thus were further confirmed by the Electrical Microscopy (EM) analysis on KO soleus muscle in which accumulation of abnormally enlarged mitochondria were found on KO soleus muscle (FIGS. 2 B and C).

b) Kyphosis and Reduced Bone Mineral Density (BMD) in 6 Month Old KO Mice.

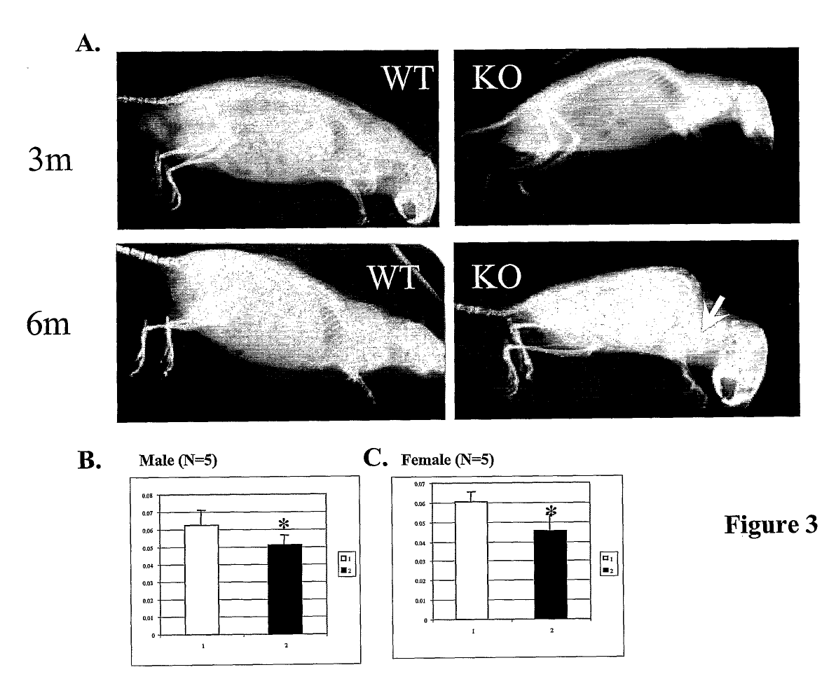

The skeletal abnormalities were also found TR4 KO mice. Radiograph of KO mice at the age of 6 months displays a prominent curvature of the spinal column (hyphosis) but not in 3-month-old KO mice (FIGS. 3B, and D), while wt mice display normal spinal column. In addition, a significant reduced BM, a sign of osteoporosis, was found in 6-month-old male and female TR4 KO mice legs (FIGS. 3 E, and F) by DEXA scanning. The osteoporosis and concomitant kyphosis exhibited by TR4 KO mice are hallmarks of aging in humans as well.

c) Ovarian Dysfunction in TR 4 KO Mice.

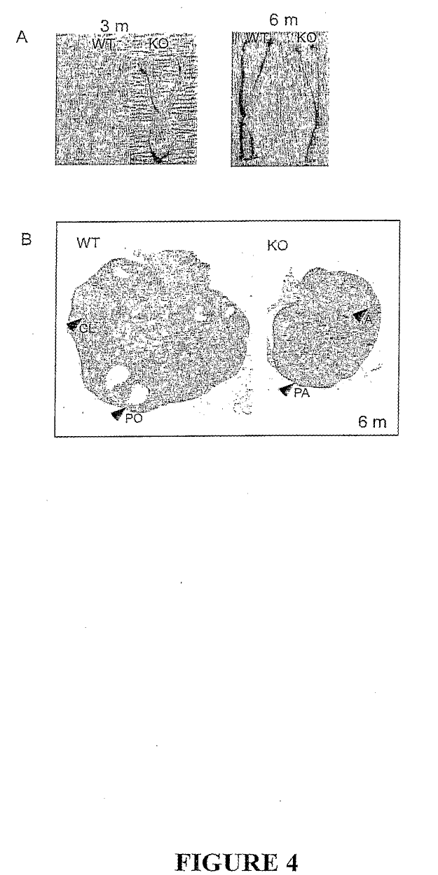

TR4 KO female mice appeared to lose fertility over time with premature ovarian failure observed in 3 of 6-month-old TR4 KO females, in which there was no active estrus cycle and complete anovulation (FIG. 4). In addition to ovarian atrophy, the uterus from KO mice displayed reduced epithelial layer and gradual structure significantly. All those phenotypes indicate TR4 KO mice develop premature aging.

d) Rapid Replicative Senescence in Tr4 KO Mice Embryonic Fibroblast (MEF) Cells, and Up-Regulation of Tr4 Expression Under Stress Condition.

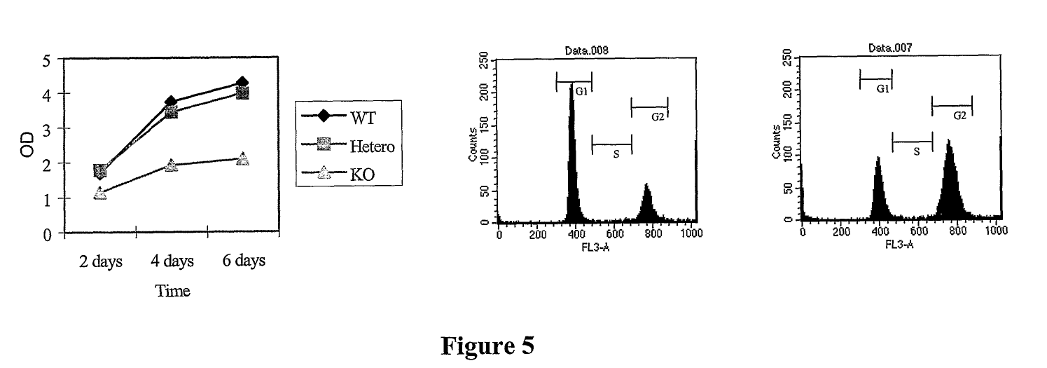

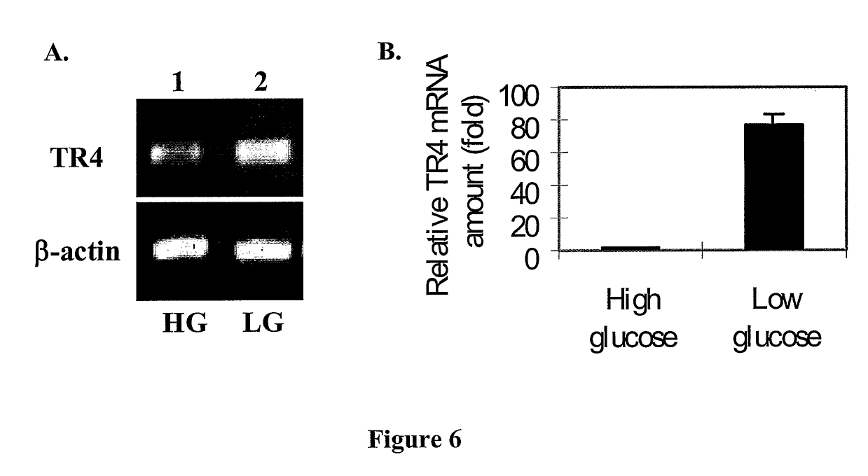

Aging refers to the biological changes occurring during a lifetime that result in reduced resistance to stress, increased vulnerability to disease, and an increased probability of death. The expression of TR4 in the liver and kidney was significantly induced when the mice were stressed with 24 hr starvation. The MEF cells from TR4 KO mice show much shorter replicative lifespan (FIG. 5), at which KO MEF cells arrest in G2/M. TR4 expressions were up-regulated when wt MEF were cultured in low glucose or treated with cobalt chloride to mimic hypoxia stress (FIG. 6). This shorter replicative lifespan and stress-induced TR4 expression indicates that TR4 functions to enhance cellular stress resistance and/or be involved in DNA repair of damaged biomolecules to protect the cells from cellular damage, and that disruption of TR4 can result in severe impairment in many aspects of biological events to shorten mice life span.

e) The Structure and Functional Study of TR4 5′-Promoter.

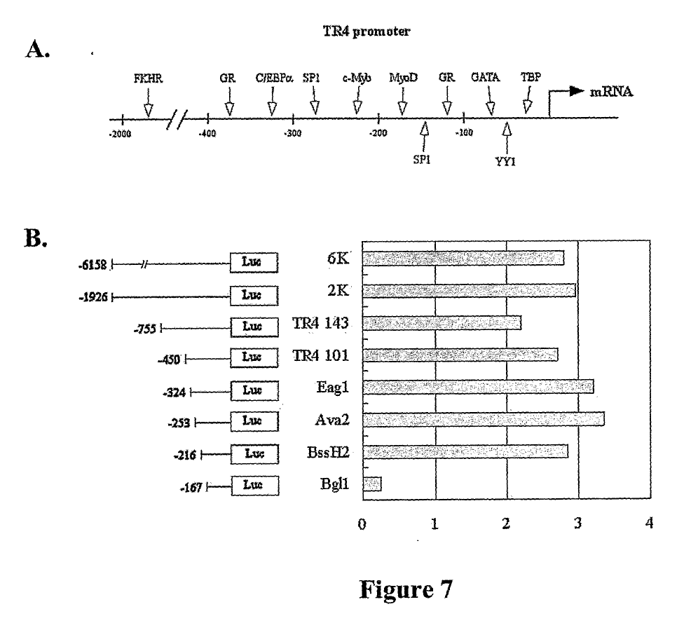

To investigate how aging related hormone or environment factors influence TR4 expression at transcriptional level, a 6.0 kb genomic DNA fragment containing the TR4 gene promoter region was cloned, sequenced, and characterized. Sequence homology search within this promoter region revealed some potential cis-acting elements which can be recognized by several transcriptional factors such as GR, C/EBPα, SP1, YY1, and MyoD. Deletion analyses and Luciferase assay showed a potential enhancer element, within 216 to 167 bp upstream of the transcriptional start site (FIG. 7), which is associated with the transcriptional of TR4 gene activity.

f) Construction of TR4RNAi.

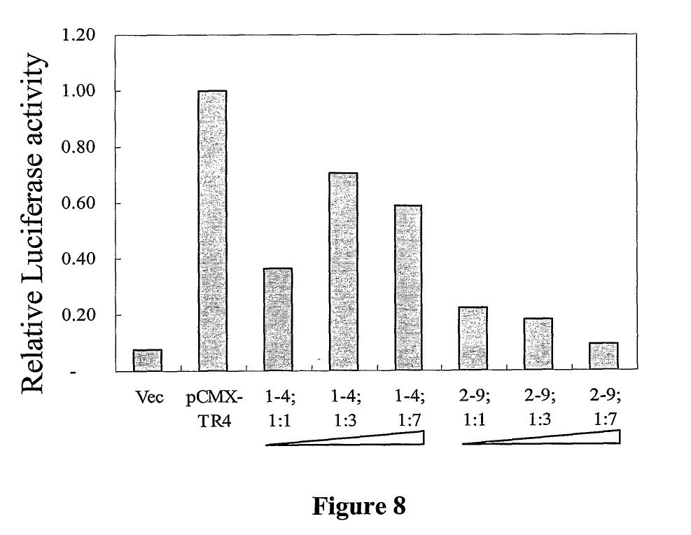

As shown in FIG. 8, two clones of TR4RNAi (1-4, and 2-9) were constructed into pSuperior.retro.puro (OligoEngine) vector, and their ability to suppress TR4-mediated TR4RE-Luc activity was tested. Clone 2-9 TR4RNAi showed a better suppression effect.

### 2. Example 2

### Determination of the Onset of Change in Multiple Organ Systems in TR4 KO Mice

TR4 KO mice developed an early onset of aging progression, which provided a model to study the initiation and progression of the aging process through monitoring the changes of multiple organ systems throughout the life span. Most importantly, changes between earlier stage vs. later stages in particular organs/systems can be identified during the aging process to identify factors operating in early or mid-life origins and consequences that occur in late stage, all of which are essential in understanding of the aging process. Organs and systems such as skin, muscle, bone, cardiovascular function, urinary function, reproductive systems, and immune systems can be studied through all segments of the life span, from neonatal (P7), before puberty (1 month), young adulthood (2-3 month), mid-age (4-6 month), mid-late (7 month to 1 yr), to late-life (over 1 year, if TR4 KO mice survive).

a) Characterization of Skeletal Muscle Defects in TR4 KO Mice.

TR4 KO mice develop mid-age onset of myopathy. Trichrome staining showed irregular, atrophy, and a large amount of mitochondria-like inclusions in TR4 KO skeletal muscle fiber, which show a decline of mitochondria function in TR4 KO mice. Initial muscle characterization is performed using hematoxylin/eosin (HE), and trichrome staining on soleus and extensor digitorum longus (EDL) muscle from at least 6 mice each of TR4 KO and wt mice at different ages in the lifespan. The inclusion bodies in the muscle fiber are further stained with anti-ubiquitin from the frozen sections. Any abnormalities detected are confirmed by electron microscopy. In addition, serum creatine kinase levels are assayed, as this enzyme is a marker for muscle stability. Some muscle marker genes are further examined. The soleus and EDL muscles are removed and frozen in liquid nitrogen cooled isopentane and sectioned at 10 μm for 1) HE and trichrome staining to analyze structure; 2) ATPase, NADH, cytochrome C oxidase, and succinate dehydrogenase enzyme staining to measure the oxidative phosphorylation (OXPHOS) status in mitochondria; and 3) ubiquitin staining to confirm the muscle inclusions. Ultrastructural analyses are then performed. Ultramicrotome sectioning and visualization with a Hitachi 7100 Electron Microscope with digital interface is then performed. The potential genes involved in TR4 KO muscle pathological changes are examined by IHC and real-time RT-PCR. The primers for genes, such as calpasin 3, fukutin, sarcoglycan, caveolin 3, dysferlin, dystroglycan are designed according to Beacon-Primer software, the total RNA and first strand DNA is prepared, and the relative expression level of genes of interest is quantified by real-time PCR.

b) Examination of Changing in the Skin and Hair in TR4 KO Mice at Different Ages.

(1) Skin

Reduced dermal thickness and subcutaneous adipose are common features in aging skin. Histological cross-sections of dorsal skin are examined from KO and wt mice at all ages. Micromorphometric measurement of sebaceous glandular sizes are also analyzed.

(2) Hair

The hair on TR4 KO mice appear to be sparser and greasier than wt mice. Hair sparseness could be a function of the ratio of hair follicle cells in the anagen (growth) phase to those in telogen (resting) phase. Hair-regrowth assays are used to test the hair regrowth ability. TR4 KO and wt mice, males and females, at all ages, are tested. Mice are shaved on the dorsal segment of skin, and the amount of hair regrowth is recorded and measured weekly.

(3) Wound Healing Test

A reduced ability to tolerate stress is a hallmark of aging, and one of such aged-related stress is delay of wound healing. To test this capacity, TR4 KO and wt mice from all ages, and genders are subjected to two 3-mm punch biopsies in the dorsal skin. The healing process is monitored and recorded every two days until the would is completely healed.

c) Examination of Reproductive Development in TR4 KO Mice.

Declining reproductive function, especially in the female reproductive system, is another indication of the aging process. In the case of TTD mutant female mice, by the age of 6 months, some mutants have complete anovulation, yet the male mice can still be fertile at the age of 7 months. Since TR4 is highly expressed in testes, it is possible that male reproductive system could also impaired in TR4 KO, therefore, both genders are examined.

In male mice, sperm count is determined, and testes, epididymes, and prostates are harvested for HE staining to confirm any pathological change. For the female reproductive system, vaginal smears are performed to determine the estrous cycle and the age that the female TR4 KO mice start to have irregular estrus cycles or cessation of cycles. The vaginal smear starts with 8-wk-old female mice from both genotypes. Each month, for 10 consecutive day (around 2 cycle in normal mice), vaginal smears are performed and continued until the TR4 KO mice show age related syndromes, and then the mice are sacrificed. The reproductive organs, such as ovary, uterus, and oviduct are harvested, weighed, and embedded in paraffin for histological and RNA/protein examinations.

d) Examination of the Potential Skeletal Abnormalities in TR4 KO Mice.

No obvious skeletal defect was found in the young TR4 KO mice in a BMD and bone morphometrial analysis. By the age of 5-6 months, some of the TR4 KO mice started to develop kyphosis and the syndrome became more severe with age, which suggested early bone loss (osteoporosis) in TR4 KO mice. BMD analysis found reduced BMD in TR4 KO mice over 6 months, which further supported this idea. Therefore, skeletal analyses of TR4 KO mice can be compared to wt mice in terms of overall skeletal structure, BMD, and bone turnover at the different ages.

Skeletal analyses are undertaken to determine structural anomalies that confirm the premature aging process in TR4 KO animals. Three age groups of mice are used for bone analysis. BMD is determined via DEXA scans of anesthetized TR4 KO and wt mice. For the bone histomorphometry analysis, animals are skinned, eviscerated, and skeletons are fixed, decalcified, and sectioned. The gross skeletal structure and ossification analyses, trabecular bone volume (TBV), and osteoblast numbers are determined. Bone remodeling markers such as TRAP, RANK, and RNAKL are measured by IHC. Serum osteocalcin, calcium, and parathyroid hormone (PTRH) levels are measured as markers associated with bone metabolism. As serum osteocalcin, calcium, and PTRH are known to correlate with osteoblast/osteoclast activity these are measured.

e) Examination of the Potential Cardiovascular Function Abnormalities in TR4 KO:

(1) Electrocardiography (ECG)

To investigate the heart conductive function, electrocardiography (ECG) is performed on wt and TR4 KO mice at different stages of age. The mice are anesthetized with an intraperitoneal injection of pentobarbital sodium (60 mg-90 mg/kg). A standard 12-lead ECG is obtained by placement of subcutaneous 22-gauge needles in each limb and rotating a chest lead to the standard locations of the precordial leads.

(2) Echocardiography

To determine if there are any defects in heart and aorta structure that can be correlated to the age-related cardiovascular failure, two-dimensional guided M-mode and Doppler echocardiography is carried out in all mice, at different ages, gender, and genotype. The mice are anesthetized using the 7.4% Nembutal Solution at a dosage of 0.005 ml/g of body weight injected intraperitoneally. Once the mouse is mildly sedated, the thoracic area is shaved from the neck caudally to the midline and latterly to the axillary areas of both forelimbs. The mice are placed on a heating pad to maintain the body temperature during the experiment in dorsolateral recumbency with the left side angled down. Prep the shaved area with a small amount of gel to smooth out any remaining hair. At the end of the ultrasound procedure, the animals are allowed to recover.

(3) Tissue Preparation for Histological Examination

TR4 KO and wt mice at different ages are sacrificed after finishing all non-invasive assays. Hearts are excised, weighed, and fixed in 10% buffered formalin and then embedded in paraffin. The sections are stained with HE and Masson's Trichrome.

f) Examination of the Immune Response in TR4 KO Mice.

Decline of the immune response, and reduced self-defense systems are associated with aging. In this aim, the immune response is examined in TR4 KO and wt mice at different ages. Splenocytes from mice are cultured and then challenged with interferon γ and TNFα, and cell proliferation (³H-thymidine incorporation), the microphage population (by flow cytometric analysis), cytokine release (ELISA), and nitric oxide (NO) (Griess reaction) production is measured, and the difference between TR4 KO and wt is compared. The related genes are analyzed by using real-time RT-PCR.

g) Determine the Gene Expression Level of the Genes Known to be Involved in Aging, and Correlate Expression Level With Age Using Q-PCR.

In order to identify factors that are affected by the aging process, Q-PCR is used to quantify the aged related gene expression changes in all segments of life in multiple organ systems as described above. The time at which TR4 KO mice start to show the aging phenotype is important to determine the differential genes expression by comparison their expression before and after this time point. Followings are the list of the genes:
A. DNA repair system (enzyme): Gadd45, Dbd2, XPD (helicase), Ku80, Xpg, Msh2, Rad23b, Xrcc1/lig3, Ercc1/Xpd, and Msh, MGMT
B. ROS scavenging proteins (enzymes)/Detoxification system: superoxide dismutase (SOD), glutathione peroxidase/reductase, and Catalase (CAT).
C. mitochondria: cytochrome C oxidases, ATP synthesis, and ribosomal proteins
D. Cell cycle checking points/tumor suppressor genes: p16, p19, p21, p27, p53, and BRCA1
E. Stress-response genes: Forkhead transcriptional factor 3 alpha (Foxo3α) and Bim
F. longevity genes: Sirt1 (or surtuin)
G. Internal control: β-actin and 18S ribosome RNA

Genes that show different mRNA levels are confirmed by their expression at the protein level using IHC and Western Blotting analysis.

### 3. Example 3

### Nitochondrial Dysfunction in TR4 KO Mice and the Status of Reactive Oxidative Stress and DNA Damage in TR4 KO Mice and their Contribution to Acceleration of Aging in TR4 KO Mice

a) Progressive Decline in Mitochondria Function Accompanies Aging.

One of the theories of mitochondria aging (MTA) is that reactive oxidative species (ROS), natural by-products of oxidative energy metabolism in mitochondria, which directly target DNA result in different lesions. The burden of ROS is largely counteracted by antioxidant defense and DNA repair systems, with inadequately repaired DNA damage eventually leading to aging. In young organisms, there are a large number of small mitochondria that provide needed ATP, however there are many large mitochondria in aged organisms. These larger mitochondria are not as bio-energetically efficient as the youthful, normal, small mitochondria. Electron microscopy examination of skeletal muscle from 6 month old TR4 KO showed enlarged and abnormal proliferation mitochondria, an indication of mitochondrial functional decline.

b) Examination of Mitochondrial DNA Integrity

The mitochondrial theory of aging (MTA) postulates that damage to mitochondria DNA (mtDNA) and organelles by free radicals leads to loss of mitochondrial function and loss of cellular energy. Mitochondria are the only cellular organelles with their own DNA. As tissues age, mtDNA mutations accumulate in individual cells; eventually in some cells, the energy generation system is seriously impaired. If mtDNA mutations occur in a significant number of cells in a tissue, the function of that tissue is compromised and consequently contributes to such age associated pathologies. Therefore, it is conceivable that TR4 KO mice can be exposed to excess ROS which results in this accumulation of mtDNA damage and accelerate aging. mtDNA integrity can be examined by examining mtDNA mutations (deletions, point mutations, gross DNA rearrangements) and their correlation with age, using PCR-based analysis.

Analysis of mitochondrial DNA integrity of several mitochondria abundance organs, brain, skeletal muscle, heart, and liver from TR4 KO mice and wt littennates can be examined. Mice, aged from young adulthood (2-3 month), mid-age (4-6 month), mid-late (7 month to 1 yr) to late-life (over 1 year, if any TR4 KO survive) are sacrificed and targeted organs are collected and immersed into liquid nitrogen immediately. Mitochondrial DNA is isolated, and extracted immediately for the following DNA mutation/deletion examinations with methods detailed in next section.

c) Detailed Methods

(1) Mitochondrial Isolation

Mitochondria are prepared from highly aerobic tissues (brain, heart, skeletal muscle, and liver) by discontinuous Percoll gradient centrifugation. The isolation buffer contains 0.32 M sucrose, 1 mM EDTA, 10 mM Tris-HCl (pH 7.4), and 5 mg/ml of BSA. The tissues are minced with scissors and then homogenized with a glass Dounce homogenizer at a 5% (wt/vol) concentration in isolation buffer. After a 1:1 dilution with a 24% Percoll buffer solution, the resulting homogenate (12% Percoll) is layered onto discontinuous density gradients (26% over 40% Percoll) and centrifuged at 30,000 times; g for 20 min at 4° C. The mitochondria-rich band at the interface between the 26 and 40% Percoll layers are collected. This fraction is pooled and diluted 1:4 with isolation buffer, and the resulting suspension centrifuged at 16,000×g for 20 min. The pellet is resuspended in isolation buffer followed by centrifugation at 7,000×g for 10 min, and the final mitochondrial pellet collected and resuspended to a final volume of 2.5 ml.

(2) DNA Extraction

Since peroxides may be present in some phenol preparations, the highest purity distilled phenol available is used for DNA extraction in order to avoid artificial oxidation of DNA during the procedure (Suliman, H. B., Carraway, M. S., Velsor, L. W., Day, B. J., Ghio, A. J., and Piantadosi, C. A. Rapid mtDNA deletion by oxidants in rat liver mitochondria after hemin exposure. Free Radic Biol Med, 32: 246-256, 2002). The mitochondrial fraction is suspended in 3 ml of TE (10 mM Tris-HCl, 1 mM EDTA) and incubated with 330 μl of 10% SDS and 400 μl of proteinase K (10 mg/ml) at 55° C. for 3 h. After incubation, the digest is extracted with 3 ml of phenol saturated with TE (1 mM EDTA, 10 mM Tris-HCl/pH 7.4) by shaking gently for 30 min. The extraction is repeated twice and then once with 3 ml of phenol/chloroform (1:1). The DNA in the aqueous phase is precipitated by adding 370 μl of 3 M sodium acetate and 8 ml of 99.5% ethanol followed by incubation for 30 min at −80° C. The precipitate is collected by centrifugation, washed with 3 ml of 70% ethanol, dried, and resuspended in TE. Purified mtDNA are stored at −20° C.

(3) Semi-Quantitative PCR(SQ-PCR)

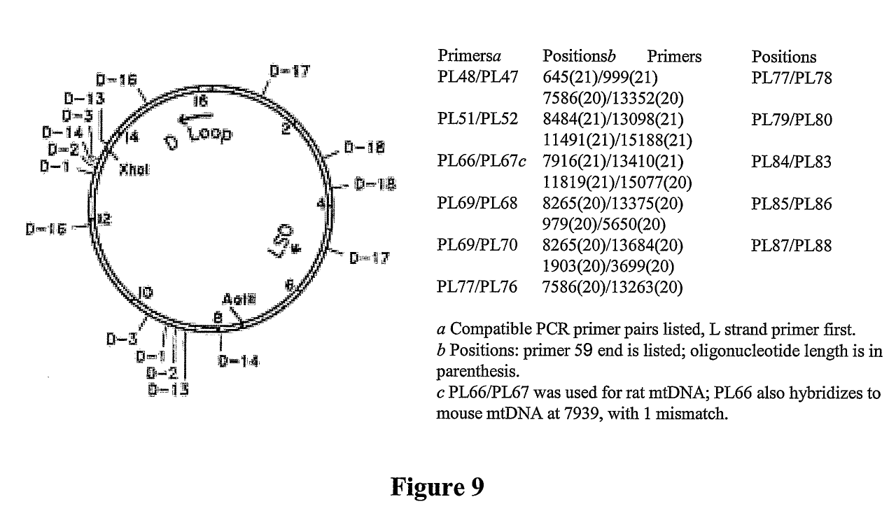

The SQ-PCR primers and strategies used are shown in FIG. 9. The primers were designed based on the sequence of rat mtDNA (Accession No. NC-001665, Genebank). The PCR approach is used under the following conditions: denaturing at 94° C. for 1 min, 55° C. for 1 min, 72° C. for 1 min for 35 cycles, followed by a final extension at 72° C. for 7 min. The PCR reaction consists of 10 mM Tris-HCl (pH 8.3), 50 mM KCl, 0.001% gelatin, 200 M of each dNTP, 2.5 mM MgCl₂, 1.25 U of AmpliTaq DNA polymerase (Perkin Elmer, Foster City, Calif., USA), 10 pmol of each primer, and 200 ng of total DNA in 50 μl reactions. Ten-microliter aliquots of the PCR products is electrophoresed through a 1% agarose gel in TAE (40 mM Tris-acetate, 20 mM sodium acetate, 1 mM EDTA) at 15 V/cm. Negative controls are included containing all of the above-mentioned PCR components except template DNA. Deletion and total mtDNA levels are quantified by product size. Experiments were performed to verify equivalent amplification efficiencies of the PCR primer products. The SQ-PCR products are quantitated using image analysis software (Bio-Rad, Hercules, Calif., USA). For each sample, the amount of deleted and total mtDNA will be determined and the deletion level expressed as a percentage of the total mtDNA level.

Direct repeats in mouse, rat, and human mtDNA

| | | | | | | | Product |
|---|---|---|---|---|---|---|---|
| DNA | | | | | | PCR | size |
| repeat | Sequence | SEQ ID NO: | Positions^{a} | Deletion size^{b} | −ΔG°₂₅ C. | primers | bp |
| D-1 | AGCCCTACTAATTAC | 10 | 9,089-9,103 | 3,867 | 24.7 | PL51/PL52 | 748 |
| | | | 12,956-12,970 | | | PL68/PL69 | 1,244 |
| D-2^{d} | TCTTTGCAGGATT | 11 | 8,844-8,896 | 4,236 | 23.6 | PL68/PL69 | (875)^{d} |
| | | | 13,120-13,132 | | | | |
| D-3 | AAGCAAATCCATAT | 12 | 9,553-9,566 | 3,726 | 24.3 | PL68/PL69 | 1,385 |
| | | | 13,279-13,292 | | | | |
| D-13 | CCCTCCTTCTAACAT | 13 | 8,677-8,691 | 4,974 | 23.0 | PL69/PL7O | 445 |
| | CCCTCCTTCCAACAT | 14 | 13,651-13,665 | | | | |
| D-14 | CAATAATAGGATTCCCAATCG | 15 | 7,964-7,984 | 5,252 | 23.1 | PL77/PL78 | (515)^{d} |
| | CAGTATTAGGATTCCTAATCG | 16 | 13,216-13,236 | | | PL76/PL77 | 424 |
| | | | | | | PL66/PL78 | 185 |
| D-16^{d} | TACCCCTATTAATATTTTTCC | 17 | 11,881-11,901 | 2,976 | 21.9 | PL79/PL78 | (721)^{d} |
| | TAACCCTAGTATTATTTTTCC | 18 | 14,857-14,877 | | | PL83/PL84 | (263)^{d} |
| D-17 | ACTAATCCTAGCCCTAGCCC | 19 | 1,094-1,113 | 3,821 | 27.4 | PL85/PL86 | 851 |
| | AATAACCCTACCCCTAGCCC | 20 | 4,915-4,934 | | | | |
| D-18^{d} | CTATCACTCACACTAGCATTAAGTCTATGA | 21 | 2,979-3,008 | 651 | 19.6 | PL87/PL88 | (1,145)^{d} |
| | CTACCCCTAACACTAGCATTATGTATGTGA | 22 | 3,630-3,659 | | | | |
| Rat | CCTGAGCCCTAATAAT | 23 | 8,103-8,118 | 4,834 | 29.3 | PL66/PL67 | 661 |
| | | | 12,937-12,952 | | | | |
| Human | ACCTCCCTCACCA | 24 | 8,470-8,482 | 4,977 | 25.2 | | |
| | | | 13,447-13,459 | | | | |

^{a}Coordinates in mtDNA
^{b}Base pairs lost if one repeat and intervening sequence are deleted
^{c}Relative stability, in kcal/mol, calculated from nearest-neighbor parameters
^{d}Predicted product not observed

d) Identification and Verification of mtDNA Deletions

The mice mitochondrial genome sequence is analyzed for perfect direct repeats since the majority of mtDNA deletions that have been identified are associated with direct repeats. The primers designed to encompass these repetitive sequences were listed as in Table I and II, according to the published paper (Tanhauser, S. M. and Laipis, P. J. Multiple deletions are detectable in mitochondrial DNA of aging mice. J Biol Chem, 270: 24769-24775, 1995). Mouse mtDNA D-1 is the longest exact match and has a high estimated stability, therefore is used as primary to examine mtDNA deletion mutants. Standard PCR amplifies a 4614-bp product from undeleted mtDNA and a 748 bp product from a D-1 deletion using primers PL51/PL52. Other repeat sequences that might produce deletions are present in mouse mtDNA and are also tested. The potential deletions, primer, and PCR product size derived from deleted mtDNA molecules are shown in Table E. The deletion products are further confirmed by Southern blotting and DNA sequencing.

e) Detection of 8-Hydroxy-2′-Deoxyguanosine (8-OHdG)

The isolated DNA is digested to nucleotides with nuclease P1 and then treated with alkaline phosphatase to liberate the corresponding nucleosides from phosphate residues. The 8-OHdG in samples are then analyzed by HPLC with the electrochemical (EC) detection method of Floyd et al. The 8-OHdG standard is obtained from Sigma Chemical (St. Louis, Mo., USA).

f) Determination of Mitochondria Dysfunction in TR4 KO

The mitochondrial electron transport chain (ETC) is the main source of cellular free radicals/oxidants, such as superoxide radical, hydrogen peroxide and hydroxyl radical. In TR4 KO mice, as mtDNA damage accumulates over the lifetime causing progressive respiratory chain dysfunction, ROS generation increases, and the functionality of the ETC enzyme complexes that produced ATP decreases dramatically and gradually produces a cellular energy crisis finally resulting in cellular failure and premature aging. The mitochondria functions in TR4 KO and wt mice can be investigated by measuring ROS (such as H₂O₂) production, oxygen consumption, ATP generation, and membrane potential.

The functions of mitochondria are assayed to determine the onset of this functional decline during the aging process and compare with wt mice. The animal age range is the same as in preceding Aim. Several aspects of mitochondrial functions are examined.

g) Preparation of Mitochondria:

Mitochondria isolation is followed as described above. They are then washed twice with ice-cold incubation buffer (each centrifugation step 10 min at 10,000×g, 4° C.). All incubations are performed at 30° C. for 30 min in a respiration buffer (110 mM mannitol, 60 mM KCl, 60 mM Tris-HCl, 10 mM K₂HPO₄, 0.5 mM Na₂EDTA, and 5 mM glutamate plus 5 mM malate as respiratory substrates, pH 7.4) with gentle shaking. Mitochondria are incubated in respiration buffer alone, or with tert-butyl hydroperoxide (50 μM) or the short acting NO donor 1,2,3,4-oxatriazolium, 5-amino-3-(3,4-dichlorophenyl)-chloride (GEA 3162, 50 μM, halftime ˜3 min) (Alexis, San Diego, Calif., USA). In some experiments, the iron chelator deferrioxamine, the putative hydroxyl radical scavenger dimethyl thiourea (DMTU), or glutathione methyl ester (GSHme) is added at a 50 μM final concentration to the mitochondrial suspensions 5 min before exposure to oxidant.

h) Determination of Mitochondrial Hydrogen Peroxide Generation

To determine the mitochondria membrane potential, the H₂O₂ generation is measured. Mitochondrial H₂O₂ production is assessed by the scopoletin oxidation method. Mitochondria (0.2 mg of protein per milliliter) is incubated in the standard respiration buffer supplemented with 10 μM scopoletin and 1 unit/ml horseradish peroxidase. Fluorescence is monitored at excitation and emission wavelengths of 365 nm and 450 nm, respectively. Calibration is performed by the addition of known quantities of H₂O₂. Each experiment is repeated at least three times with different mitochondrial preparations (Tieu, K., Perier, C., Caspersen, C., Teismann, P., Wu, D.C., Yan, S. D., Naini, A., Vila, M., Jackson-Lewis, V., Ramasamy, R., and Przedborski, S. D-beta-hydroxybutyrate rescues mitochondrial respiration and mitigates features of Parkinson disease. J Clin Invest, 112: 892-901, 2003).

i) Determination of Membrane Potential (Δφm)

Mitochondrial membrane potential is measured by using the fluorescence signal of the cationic dye safranine O, which is accumulated and quenched inside energized mitochondria. Mitochondria (0.2 mg protein/ml) are incubated in the standard respiration buffer supplemented with 10 μM safranine. FCCP (5 μM) is used as a positive control to collapse Δφm. Fluorescence is detected with an excitation wavelength of 495 nm (slit 5 nm) and an emission wavelength of 586 nm (slit 5 nm) using a Hitachi (Tokyo, Japan) model F-3010 spectrofluorometer. In different laboratories it has been shown that the addition of ADP promoted a 5-10% decrease of the Δφm.

j) Oxygen Consumption Measurements

Oxygen uptake is measured in an oxymeter fitted with a water-jacketed Clark-type electrode (Yellow Springs Instruments Co., model 5300). Mitochondria (0.2 mg/ml) are incubated with 1.5 ml of the standard respiration buffer described above. The cuvette is closed immediately before starting the experiments. Each experiment is repeated at least three times with different mitochondrial preparations. Respiratory control ratio values are obtained with isolated mitochondria by using both pyruvate and malate as complex I substrates or succinate as a complex II substrate and is in good agreement with previous reported values (da-Silva, W. S., Gomez-Puyou, A., de Gomez-Puyou, M. T., Moreno-Sanchez, R., De Felice, F. G., de Meis, L., Oliveira, M. F., and Galina, A. Mitochondrial bound hexokinase activity as a preventive antioxidant defense: steady-state ADP formation as a regulatory mechanism of membrane potential and reactive oxygen species generation in mitochondria. J Biol Chem, 279: 39846-39855, 2004.)

k) Measurement of ATP Generation

Isolated mitochondria are suspended in respiration buffer and then stored at −70° C. before use. The content of ATP in the mitochondria are measured using the ATP Bioluminescence assay kit (Roche Molecular Biochemicals) and detailed protocol are followed according to manufacturer's instructions. Light emitted from luciferase-mediated reaction is captured in a tube luminometer and calculated from a log-log plot of the standard curve of known ATP concentrations (as provided by kit).

l) Measurement of Changes of Complex Activity in KO with Different Age

The mitochondria respiratory chain complexes' activity from TR4 KO mice are compared at different ages, as well as age-matched wt mice as controls. Spectrophotometric assays is used to measure the activity of each of the components of the electron transfer chain: complex I (NADH: ubiquinone oxidoreductase); complex II (succinate: ubiquinone oxidoreductase); complex III (ubiquinol: cytochrome C oxidoreductase); and complex IV (cytochrome c oxidase). Mitochondria isolated from organs (liver and brain) are suspended in mitochondria isolation buffer and stored at −70° C. until used. Enzyme activity assays are carried out as described (Taylor, R. W., Chinnery, P. F., Turnbull, D. M., and Lightowlers, R. N. Selective inhibition of mutant human mitochondrial DNA replication in vitro by peptide nucleic acids. Nat Genet, 15: 212-215, 1997.). Briefly, complex I is measured as decrease in absorbance at 340 nm due to the oxidation of NADH with a reference wavelength of 425 nm. Complex II measurements follow the succinate-dependent reduction of 2,6-dichlorophenol-indophenol at 600 nm. Complex III is measured by following the reduction of cytochrome c (III) by ubiquinol at 550 nm with a reference wavelength of 580 nm. Complex IV is also measured at 550 nm (with a reference wavelength of 580 nm) following the oxidation of cytochrome c (II). All enzyme activities are corrected for the amount of protein in the preparation (Menzies, F. M., Cookson, M. R., Taylor, R. W., Turnbull, D. M., Chrzanowska-Lightowlers, Z. M., Dong, L., Figlewicz, D. A., and Shaw, P. J. Mitochondrial dysfunction in a cell culture model of familial amyotrophic lateral sclerosis. Brain, 125: 1522-1533, 2002).

Measuring protein expression levels of respiratory chain complex showing altered biochemical activity. Western Blotting is used to determine whether any of the changes in activity of respiratory chain complexes during the aging process is accompanied by alternations in the level of overall protein expression. Complex II can be detected using an antibody to iron/sulphur subunit of succinate dehydrogenase and complex IV using an antibody to subunit 1 of cytochrome c oxidase. The membrane is stripped and re-probed with actin antibody as loading control.

### 4. Example 4

### The Molecular Mechanisms of Rapid Replicative Senescence in Tr4 KO MEF Cells and its Contribution to Accelerated Aging

MEF rapid senesce is believed to be the result of severe oxidative stress which induces extensive DNA damage and/or chromosomal aberrations and is a landmark of aging cells. TR4 KO MEF cells display a rapid replicative senescence, at which TR4 KO MEF cells arrest at G2/M phase after four population doublings (P4), indicating that TR4 KO MEF cells fail to overcome replicative senescence that is caused by oxidative stress. MEF cells derived from TR4 KO and wt mice can be examined to determine the mechanisms underlying the replicative senescence and determine its contribution to accelerated aging in mice. MEF cells are challenged with DNA-damage inducers, such as hydrogen peroxide and UV, and then ROS status, the degree of DNA damage, DNA repair ability, DNA replication, and cell survival is measured and compared. Viral TR4 infection is used to rescue the defects in TR4 KO MEF to confirm the roles of TR4. The known genes related to the stress-response, cell survival, and DNA damage/repair systems are compared between TR4 KO and wt MEF cells. In addition, microarray analysis is used to identify the TR4 targeted genes, which are responsible for the TR4 KO MEF rapid replicative senescence.

a) Characterization of TR4KO MEF Cells.

Oxidative stress severely limits the replcaitive lifespan of murine fibroblast. To study the underlying mechanisms of the rapid replicative senescence found in TR4 KO MEF cells, and its contribution to the premature aging, MEF cells are characterized. The alternations of their sensitivity to the oxidative stress is compared among MEF cells from TR4KO, heterozygote, and wild type. Briefly, MEF cells are generated from 13.5 to 14.5 ED embryos, after confirmation of genotyping, MEF cells with different genotypes are seeded and challenged with or without oxidative stress, such as H₂O₂ in different dose (from 100 μM to 1 mM), and their cell cycle profile examined, cell proliferation rate and chromosome fragmentation analyzed. Due to the rapid senesce in TR4 KO MEF cells, only the MEF cells before P4 are used. To further confirm TR4 role in regulation of cell survival, TR4 RNAi and TR4-viral overexpression system are applied. The detailed methods for each experiment are described in next section.

b) Detailed Methods

(1) Cell Cycle Profile

The Flow Cytometry can be used to determine cell cycle profiles in MEF cells from TR4 KO and wt mice. Briefly, the cells are harvested at different passages (from P1 to P4), and then fixed overnight with 70% ethanol. The next day, fixed cells are stained with Propidium Iodide to determine the DNA content, and cell cycle profiles are obtained by flow cytometry.

(2) Cell Proliferation Assay

MEF cells from TR4 KO and wt mice are cultured and tested before passage 4 (P4). Cells (500-1000) are seeded. After they attach, the cells are treated with 100 μM to 1 mM H₂O₂ for 2 hrs, then are replaced with serum containing culture medium. The cells then are harvested to determine the proliferation rate at day 1, 2, 4, and 6. The cell proliferation rate (or DNA replication capacity) is determined by ³H-tymidine incorporation and MTT assays. H₂O₂-treated surviving cells is calculated as the ratio of cell number in H₂O₂-treated group as to non-treated group.

(3) Cytogenesis Analysis

To examine the chromosome fragments that occurs during the oxidative stress and are seen in senescent cells, MEF cells are treated with colcemid (0.1 μg/ml) for 4 hs, trypsinized, and centrifuged at 120 g for 8 min. After hypotonic swelling in sodium citrate (0.03 M) for 25 min at 37° C., the cells are gradually fixed in methanol: acetic acid (3:1). Cell suspensions is dropped onto clean wet slides and dried overnight. Fluorescence in situ hybridization (FISH) with a Cy-3-labelled probe (Applied Biosystem, Foster City, Calif.) is performed. Cy3 and DAPI image from 50 metaphases is acquired with fluorescence microscope and analyzed. The telomeric Cy3 image is superimposed on the DAPI image to Accurately score chromosome aberrations using image software package.

(4) Examination of MEF Cells for Response to Oxidative Stress

MEF cells from three genotypes (TR4 KO, heterozygote, and wt) are treated with different dose of H₂O₂ (100 nM, 250 nM, 500 nM and 1 μM) for two hrs, and then replaced with fresh medium. ROS status and detoxification ability is then measured to monitor the anti-oxidant scavenge responses. The degrees of DNA damage and DNA repair capacity is measured. Finally, the cell survival and/or the proliferation is determined. To confirm further for TR4 roles in response to oxidative stress, TR4 RNAi and TR4-viral overexpression systems are applied.

(5) Measurement of Intracellular ROS by Flow Cytometry

Production of intracellular ROS is detected by flow cytometry using DCFH-DA. The MEF cells are cultured in 60-mm tissue-culture dishes. The culture medium is replaced with new medium when the cells are 80% confluent. Following H₂O₂ incubation, cells are treated with 10 μM DCFH-DA for 30 min in the dark, washed once with PBS, detached by trypsinization, collected by centrifugation, and suspended in PBS prior to flow cytometry. The intracellular ROS, as indicated by the fluorescence of dichlorofluorescein (DCF), is measured with a Becton-Dickinson FACS-Calibur flow cytometer (Hsieh, T. J., Liu, T. Z., Chia, Y. C., Chem, C. L., Lu, F. J., Chuang, M. C., Mau, S. Y., Chen, S. H., Syu, Y. H., and Chen, C. H. Protective effect of methyl gallate from Toona sinensis (Meliaceae) against hydrogen peroxide-induced oxidative stress and DNA damage in MDCK cells. Food Chem Toxicol, 42: 843-850, 2004).

(6) Measurement of Redox (Gth) Detoxification Assay

Total GSH content of the MEF cells are determined using a Cayman GSH assay kit (Cayman Chemical Co., Ann Arbor, Mich.). The assay uses a carefully optimized enzymatic recycling method using GSH reductase whereby the sulfhydryl group of GSH reacts with 5,5′-dithio-bis-2-nitrobenzoic acid and Ellman's reagent producing a yellow colored 5-thio-2-nitrobenzoic acid (TNB). The mixed disulfide, GSTNB (between GSH and TNB), which is concomitantly produced, is reduced by GSH reductase to recycle GSH and produce more TNB. The rate of TNB production is directly proportional to this recycling reaction, which is in turn directly proportional to the concentration of GSH in the sample. Thus, measurement of TNB at 405 or 412 nm provides an accurate estimate of GSH present in the sample. It should be noted that oxidized GSH is converted to GSH by GSH reductase in this system, which consequently measures total GSH (Zhou, X. J., Vaziri, N. D., Wang, X. Q., Silva, F. G., and Laszik, Z. Nitric oxide synthase expression in hypertension induced by inhibition of glutathione synthase. J Pharmacol Exp Ther, 300: 762-767, 2002).

(7) DNA Damage Management

(a) Measurement of DNA Single-Strand Breaks

A DNA precipitation assay is used for DNA-strand-breaks detection. Confluent MEF cells in 35-mm dishes are labeled in the presence of 0.25 μCi/ml [³H]methylthymidine for 24 h. The cells are thoroughly washed with PBS, and supplied with 2 ml of PBS with the indicated additions. After treatment, the cells are washed with PBS and lysed in a 10 ml disposable tube with 1 ml of lysis buffer (10 mM Tris/HCl/10 mM EDTA/50 mM NaOH/2% SDS, pH 12.4) followed by addition of 1 ml of 0.12 M KCl. The lysate is incubated for 10 min at 65° C. followed by a 5 min cooling-and-precipitation period on ice. A DNA-protein K-SDS precipitate is formed under these conditions, from which low-molecular-mass broken DNA is released. This DNA is recovered in the supernatant from a 10 min centrifugation at 200 g, 10° C., and transferred into a liquid scintillation vial containing 1 ml of 50 mM HCl. The precipitated pellet (intact double-stranded DNA) will be solubilized in 1 ml of water at 65° C., the tube is rinsed with 1 ml of water, and 8 ml of scintillation fluid is added to each vial. The amount of double-stranded DNA remaining is calculated for each sample by dividing the d.p.m. value of the pellet by the total d.p.m. value of the pellet+supernatant and multiplying by 100. The results representing the extent of DNA damage is calculated as (Dt/Dc)×100, where Dt represents double-stranded DNA in treated cells and Dc represents double-stranded DNA in the respective control cells. In control cells (cells incubated in Ca²⁺-containing or Ca²⁺-free/EGTA), the level of total double-stranded DNA is around 75%. Pretreatment with the various chelators did not affect this level (Jornot, L., Petersen, H., and Junod, A. F. Hydrogen peroxide-induced DNA damage is independent of nuclear calcium but dependent on redox-active ions. Biochem J, 335 (Pt 1): 85-94, 1998).

(b) Comet Assay

An Fpg-FLARE (fragment length analysis using repair enzymes) comet assay kit is used in accordance with the manufacturer's instructions (Trevign, Ginthersberg, Mo.). This kit specifically detects oxidative DNA lesions such as 8-oxo-2-deoxyguanosine and formamidopyrimidines. Images of 50 randomly chosen nuclei per sample is captured using a CCD camera coupled to an epifluorescence microscope. Comet tail lengths are measured using the comet macro from NIH public domain image analysis program.

(8) DNA Repair Capacity Measurement:

(a) End-Joint Assay

pUC19 carries an ampicillin resistance gene and the lacZ gene. A double strand DNA break (DSB) is generated in purified pUC19 DNA at the BamHI restriction endonuclease site, which is disrupted in the lacZα gene. To assess the DSB repair efficiency, 50 μg of protein extract from each genotype is incubated for 1 h at 30° C. with 1 μg of linear pUC19 in a 50 μl reaction mixture of DSB repair buffer. The reaction is stopped and DNA purified. Transformants are selected on LB plates containing 100 μg/ml ampicillin and 40 μg/ml X-gal. The total colony number representing the DSB repair ability and blue colony number representing DNA repair fidelity is measured (Kulesza, P. and Lieber, M. R. DNA-PK is essential only for coding joint formation in V(D)J recombination. Nucleic Acids Res, 26: 3944-3948, 1998).

(b) DNA Glycosylase Assay

Incision of uracil, 5-OH-uracil, or 5-OH-cytosine in 30-mer oligonucleotides was measured essentially as described in Nyaga, S. G. and Bohr, V. A. Characterization of specialized mtDNA glycosylases. Methods Mol Biol, 197: 227-244, 2002. Reactions (20 μl) contain 70 mM HEPES (pH 7.5), 1 mM EDTA, 1 mM DTT, 75 mM NaCl, 0.5% BSA, 90 fmol of oligonucleotide, and 25 μg of protein extract. Assays are incubated at 37° C. for 1 h. Incision of 8-oxodG from a 28-mer oligonucleotide is measured. Reactions (20 μl) contain 40 mM HEPES (H 7.6), 5 mM EDTA, 1 mM DTT, 75 mM KCl, 10% glycerol, 88.8 fmol of oligonucleotide, and 50 μg protein extract. Reactions are incubated for 2-6 hrs at 32° C. then terminated by adding 5 μg of proteinase K (PNK) and 1 μl of 10% SDS and incubating at 55° C. for 30 min. DNA is precipitated by addition of 1 μg glycogen, 4 μl of 11 M ammonium acetate, 60 μl of ethanol, and overnight incubation at 20° C. Samples will be centrifuged, dried, suspended in 10 μl of formamide loading dye (80% formamide, 10 mM EDTA, 1 mg/ml xylene cyanol FF, and 1 mg/ml bromophenol blue) and loaded onto a 20% acrylamide and 7 M urea gel. Substrate and product DNA is resolved by electrophoresis at 15 W for 1 h 10 min. Gels is visualized by PhosphorImager and analyzed by using ImageQuant™ (Molecular Dynamics). Incision activity is determined as the intensity of product bands relative to the combined intensities of substrate and product bands.

(c) Base Excision Repair Synthesis Assays (BER)

Repair synthesis of an uracil containing duplex is measured in protein extracts from KO or wt MEF cells. Reactions contain 40 mM HEPES, 0.1 mM EDTA, 5 mM MgCl₂, 0.2 mg/ml BSA, 50 mM KCl, 1 mM DTT, 40 mM phosphocreatine, 100 μg/ml phosphocreatine kinase, 2 mM ATP, 40 μM dNTPs, 4 μCi ³²P-dCTP, 3% glycerol, and 120 ng double-strand oligonucleotide, in 20 μl. Nuclear or mitochondrial extract (40 μg) is added, and reaction mixtures are incubated at 37° C. for 15, 30, or 60 min, followed by addition of 1 unit of T4 DNA ligase. Reactions are terminated by adding 5 μg of proteinase K (PNK) and 1 μl of 10% SDS and incubating at 55° C. for 30 min. Substrate and product DNA is resolved by electrophoresis at 15 Walts for 1 h 10 min. Gels are visualized by PhosphorImager and analyzed by using ImageQuant™ (Molecular Dynamics). Quantification of repair synthesis activity is performed by comparing the average relative intensities of the ³²P-dCTP-containing 30-mer in KO vs. wt protein extracts.

(d) Measurement of Protective Effects from DNA Damage

To determine the protective effects of cellular proteins from either TR4 KO or wt mice MEF cells, plasmid DNA (pUC19 DNA; 1.25 μg) is added with protein extraction from TR4 KO and wt, and TR4 KO rescued by TR4-viral transfected MEF cells (10 μg, PBS as control). Then 5 mM H₂O₂ and 0.33 mM FeSO4 are added and incubated at 37° C. for one hr. The reaction mixture is electrophoresed on a 0.8% agarose gel, and supercoiled (SC) and open circle (OC) bands are quantitated and analysis using Quantity One program (BioRad). Protective effects of scavenging proteins from MEF cells is measured as percentage of SC DNA which represents the inhibition effect of .OH-induced plasmid DNA breaks as described previously (Tian, B., Wu, Y., Sheng, D., Zheng, Z., Gao, G., and Hua, Y. Chemiluminescence assay for reactive oxygen species scavenging activities and inhibition on oxidative damage of DNA in Deinococcus radiodurans. Luminescence, 19: 78-84, 2004).

(e) Luciferase-Based DNA Repair Measurement

Cells are transfected with 0.5 μg of the CMV-luciferease damaged by 5000 J/m² of UV irradiation or 200 μM H₂O₂ to induce DNA damage and 0.1 μg of the undamaged CMV-renilla, and treated with virus supernatants of the pBabe-TR4 transfected cells or the pBabe transfected cells. 24 hours after transfection, luciferase assays is performed. DNA repair was assayed by the luciferase activities. CMV-luciferease luciferease activities are normalized to that of CMV-renilla. Fold repair is calculated by dividing the normalized luciferase activities by that of the empty vector. Detailed as described previously (Tran, H., Brunet, A., Grenier, J. M., Datta, S. R., Fornace, A. J., Jr., DiStefano, P. S., Chiang, L. W., and Greenberg, M. E. DNA repair pathway stimulated by the forkhead transcription factor FOXO3a through the Gadd45 protein. Science, 296: 530-534, 2002.)

(f) Q-PCR-based DNA Repair Measurement

Cells are transfected with 0.5 μg of the pBluescript vector (Stratagene) damaged by 5000 J/m² of UV irradiation, or 200 μM H₂O₂, 0.1 μg of the undamaged pGL3-Basic vector (Promega), and 0.4 μg of the pBabe-TR4 construct or 0.4 μg of the pBabe/pure (an empty vector). DNA repair is assayed by quantitative real-time PCR using T3 and T7 primers for the pBluescript vector, and GL2 and RV3 primers for the pGL3-Basic vector. pBluescript PCR quantities are normalized to pGL3-Basic PCR quantities. Fold repair is calculated from the normalized PCR quantities divided by those of the empty vector.

(9) Rescue MEF KO Defects in Response to Oxidative Stress by Restoring Tr4.

In order to further prove that loss of TR4 indeed increased cell oxidative stress, and accumulation of oxidative stress consequently caused DNA damage, and un-repaired DNA damages then result in cell senescence. KO MEF cells are transfected with pBabe-TR4 to see if restoring TR4 can rescue this rapid cell senescence. To achieve high transfection efficiency retroviral-mediated gene transfer is used to deliver TR4 gene into MEF cells.

(10) Retroviral-Mediated Gene Transfer

To restore TR4 expression in TR4 KO MEF cell, pBabe-hTR4 is used for retroviral infection. Ecotropic packaging cells is plated for 24 hrs and then transfected with SuperFect (Quigen) with pBabe-pur/2 or pBABE-TR4. After 48 hs, the viral containing medium is filtered (0.45 mM filter, Millipore) to obtain viral-containing supernatant. Targeted MEF cells are plated and the culture medium is replaced with a mix of the viral-containing supernatant and culture medium, supplemented with 4 μg/ml polybrene, and the cells are incubated at 37° C. MEF cells infected with the empty vector (pBABE-puro) is used as control.

(11) Identification of Tr4 Targets in Response to Oxidative Stress-Related DNA Repair

The known genes related to the stress-response, cell survival, DNA damage/repair systems are compared between TR4 KO and wt MEF cells. The genes that are examined are listed above. In addition, microarray analysis is applied to identify the TR4 targeted genes, which are responsible for the TR4 KO MEF rapid replicative senescence. The MEF cells from TR4 KO and wt at the same passage number are compared for the change of gene profile. MEF cells are treated with H₂O₂ (or vehicle as control) for two hrs, and then medium replaced with fresh medium, then cells are harvested at 0, 1, 3, and 8 hrs after treatment. Transcriptional profiles are compared, and a clustering algorithm is applied to identify genes regulated by TR4 after DNA-damage induction. The genes displaying the most distinct differences, which are screened from known genes and/or microarray, are further confirmed by Real-time Q-PCR and Western blotting analysis.

(12) Microarray Assay

MEF cells from KO and wt mice are treated with vehicle alone, or with H₂O₂ to induce the oxidative stress, and then the cells are harvested for RNA preparation. RNA is then subjected to analysis using Affymetrix (Santa Clara, Calif.) HG-U95A high-density oligonucleotide array. Microarray data from MEF samples is compared from wt vs TR4 KO mice, under conditions of oxidative stress vs sham-treatment and search for the 10 most distinct differentially expressed genes, and the genes that are involved in transcription or DNA repair system are the priority. The candidates are confirmed using Real-time Q-PCR, and Western blotting if antibodies are available. The candidates that test positive are further analyzed for their regulation. The transcriptional regulation of targeted genes by TR4 is further tested using the promoter containing reporter tests. Putative TR4 response elements are analyzed by EMSA assay.

### 5. Example 5

### Identification of the Signaling Pathways which Modulate Tr4 Expression and Activity that Results in the Regulation of TR4-Meidated Aging Process

TR4 is the first steroid nuclear receptor shown to have a biological function closely linked to aging, therefore identification of signaling pathways that modulate TR4 activity is important for understanding the fundamental actions of TR4, and how this relates to the aging process. Generally, steroid hormone receptor activity can be altered by either agonists (or ligands) or by modulating the expression of receptor to change the receptor sensitivity. Aging-related hormones, like dehydroepiandrosterone (DHEA), DHEAS, thyroid, sex hormones and cortisol can modulate TR4 activity. TR4 targeted genes and TR4 functions (its roles in DNA damage, repair, and cell proliferation response to H₂O₂) are examined by using the MEF cell system. On the other hand, the modulation of TR4 activity can also be achieved via regulation of TR4 expression level, therefore study of the 5′-TR4 reveals how environment factors such as stress can influence TR4 activity. It was found that stresses like starvation and hypoxia can induce TR4 expression, which implied TR4 is a stress-sensor and is able to respond to environmental threats and protect individuals from damage. Therefore, 5′-TR4-promoter Luc-reporter system can be used, as well as MEF cells from TR4 KO and wt mice to study how those potential environment factors and aging related hormone modulate TR4 activity. A 6 kb of TR45′-flanking region and its serial deletions have been cloned and constructed into Luciferase reporter genes ready for testing.

The aging hormones, such as DHEA, DHEAS, thyroid, and sex hormones (testosterone, progesterone, and estrogen) decline with age. In contrast, glucocorticoid steroid hormones show rising blood levels with age (Djordjevic-Markovic, R., Radic, O., Jelic, V., Radojcic, M., Rapic-Otrin, V., Ruzdijic, S., Krstic-Demonacos, M., Kanazir, S., and Kanazir, D. Glucocorticoid receptors in ageing rats. Exp Gerontol, 34: 971-982, 1999; Barrou, Z., Charru, P., and Lidy, C. Dehydroepiandrosterone (DHEA) and aging. Arch Gerontol Geriatr, 24: 233-241, 1997). Since lack of TR4 results in acceleration of aging in mice, and rapid senescence in MEF cell, and TR4 is a nuclear orphan receptor which shares structure similarity with other steroid hormone receptors without known ligand being identified, therefore it is therefore likely these aging hormones are able to either activate/repress TR4 activity or induce/repress TR4 expression (similar situation like DHEA or androstendiol that can weakly activate androgen receptor although they are not androgen receptor authentic ligands). The alterations of TR4 target genes, DNA damage, DNA repair, and cell proliferation from aging hormone-treated MEF cells from TR4 KO or wt mice can be examined. In addition, the TR4-viral transfer system can be applied to restore TR4 into TR4 KO MEF cells, and TR4 RNAi to knockdown TR4 expression in wt MEF cells and to confirm all TR4-mediated effects of these aging hormones. As shown in FIG. 8, several TR4 RNAi's that can suppress TR4-mediated transcriptional activities have been obtained which can be used in this study.

a) Environmental Stress can Influence Tr4 Activity

Oxidative free radicals induce DNA damage which is believed to be involved in the aging process. Cells are treated with H₂O₂ to generate oxidative stress and then examine the alterations in TR4 expression in mRNA, as well as protein levels. Different doses of H₂O₂ (from 100 μM to 1 mM) are used to treat wt MEF cells, or mice Hep1-6 cells for two hrs, and then medium is replaced with fresh culture medium. The cells are harvested at different times (30 min, 2, 4, 12 up to 24 hrs) and RNA and protein is extracted. The TR4 expression level is analyzed by Q-PCR and Western Blotting. To clarify if new protein synthesis is needed for the up-regulation of TR4, cyclohexamide, a protein synthesis inhibitor is applied. Through this, whether stress-stimulated TR4 is regulated in the transcriptional or translational levels can be determined. Based on the findings, TR4 protein was up-regulated under stress stimulation, which suggested that TR4 promoter contains a stress-responsive element (SRE) corresponding to the stress. A 6 kb of TR45′-flanking region and its serial deletion has been cloned and constructed into Luciferase reporter genes. The transcriptional activity can be tested on the 5′-TR4-Luc and its serial deletions upon challenges such as DNA damage agents (oxidative stress (H₂O₂-treatment), UV-light, and radiation), and low nutrition (low glucose and low serum) to determine the SRE. Furthermore, MEF cell from TR4 KO vs wt mice can be applied to confirm if these stress-induced agents (DNA damage and low nutrition) can modulate TR4 activity only in wt MEF cells, but not in KO MEF cells.

b) Detailed Methods

(1) Real-Time Q-PCR of TR4 mRNA Expression

MEF and Hep1-6 cells are treated different doses of H₂O₂ (from 100 μM to 1 mM) for two hrs and then medium is replaced with fresh culture medium and cultured for 48 hours. The RNA samples are obtained by Trizol reagents, and total RNA is converted into first strand cDNA by SuperScript II reverse transcriptase (Invitrogen). Primers for amplification of TR4 are designed by the Becon Primer Designs software. Q-PCR (or Real-time PCR) is performed using Bio-Rad iQ cycler. CT values are calculated and normalized to the level of the housekeeping gene β-microglobulin. Relative gene expression is calculated according to 2^{-ΔΔCT} from three independent experiments.

(2) Transfection Assay and Luciferase Assays

The 6 kb and serial deleted constructs with Luc reporter is transfected into CV1 cells, and then cells are treated with H₂O₂ (250 μM), as well as other stresses. The region(s) that lose the response to H₂O₂-induced 5′-TR4-Luc activity are potential SREs. More stress challenge, such as UV-irradiation, ionizing radiation, and low glucose are applied to determine the SREs within the TR45′-promoter. The putative SRE regions which are critical for stress response are further narrowed down by site directed mutagenesis. The goal is to identify the minimal regions, around 30-50 bp, responsible for the stress-induced TR4. Transient transfection are performed by using SuperFect according to the manufacturer's suggested procedure (Qiagen). After transfection, cells are treated with 250 μM H₂O₂ for two hrs, and then medium is replaced with cultured medium for 48 hours. Cell lysates are prepared and the luciferase activity is normalized for transfection efficiency using pRL-CMV as an internal control. Luciferase assays are performed using dual-luciferase reporter system (Promega).

(3) Site-Directed Mutagenesis of Potential SRE

If putative SREs identified from the serial deletionTR4 5′-promoter study contain some known cis-acting elements, the sequences in these cis-acting elements is mutated by using QuickChange Site-Directed mutagenesis kit (Strategene). If the regions contain no known cis-acting elements, that region can be mutated every 15-20 bp to narrow down the minimal regions for TR4 activation.

### 6. Example 6

### Accelerated Aging with Impaired Genomic Stability Maintenance in Mice Lacking TR4 Orphan Nuclear Receptor

a) Premature Aging Phenotype.

TR4^{-/-} mice, in general, have shorter lifespan (average <12 month, compared with >2 years from wild type (TR4^{+/+}) mice and more than 95% of the TR4^{-/-} mice (67 out of 70 TR4^{-/-} mice) won't live over one year of age, without an obvious cause of death. Adult TR4^{-/-} mice display growth impairments, including reduction of body weight and hypoglycemia. By 6 months it was noticed that TR4^{-/-} mice acquire “aged” appearances in which some of TR4^{-/-} mice have grey and greasy hair (FIG. 1). To note that the serum albumin levels, and food intakes/body weight are similar in TR4^{-/-} and TR4^{+/+} mice which indicates that aged-related phenotypes were not likely due to nutritional problems, prompting us to conduct a more systematic analysis of parameters involved in premature aging.

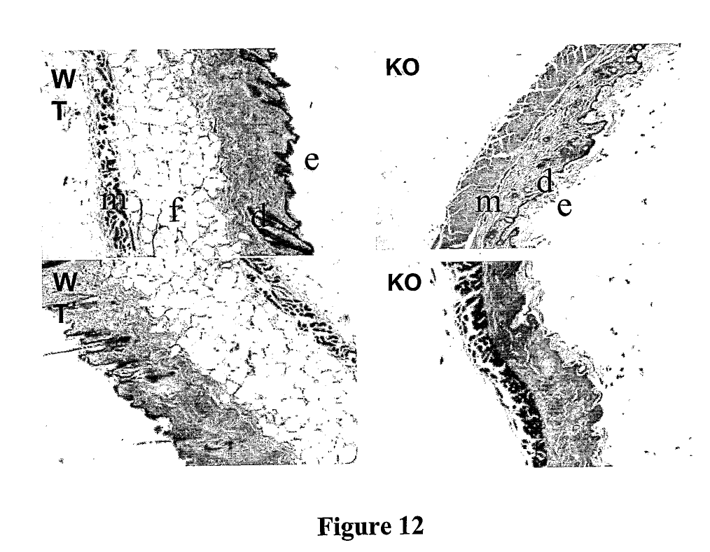

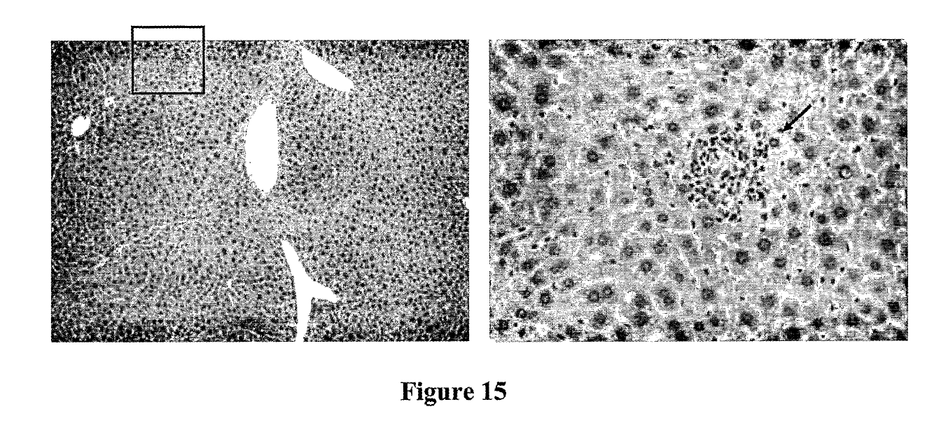

Two markers for aging skin in humans are reduced dermal thickness and subcutaneous adipose. Histological cross-sections of dorsal skin revealed no significant differences in the skin structure of 3-month-old TR4^{+/+} and TR4^{-/-} mice, but significant reduction of dermal thickness and absence of subcutaneous adipose cells were seen in 6-month-old TR4^{-/-} mice while TR4^{+/+} retained normal skin structure (FIG. 12). In addition, TR4^{-/-} mice show an extramedullary haematopoiesis in the liver (FIG. 15) with an indication of anemia that is commonly found in aging mice liver.

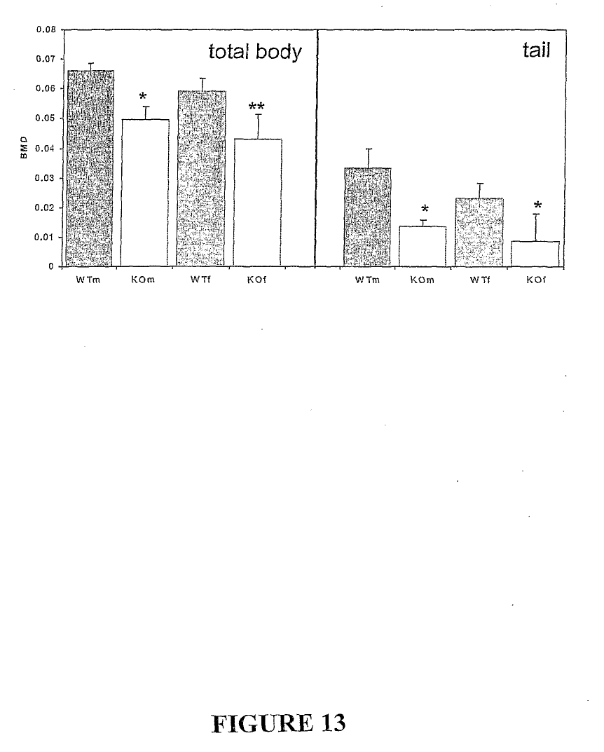

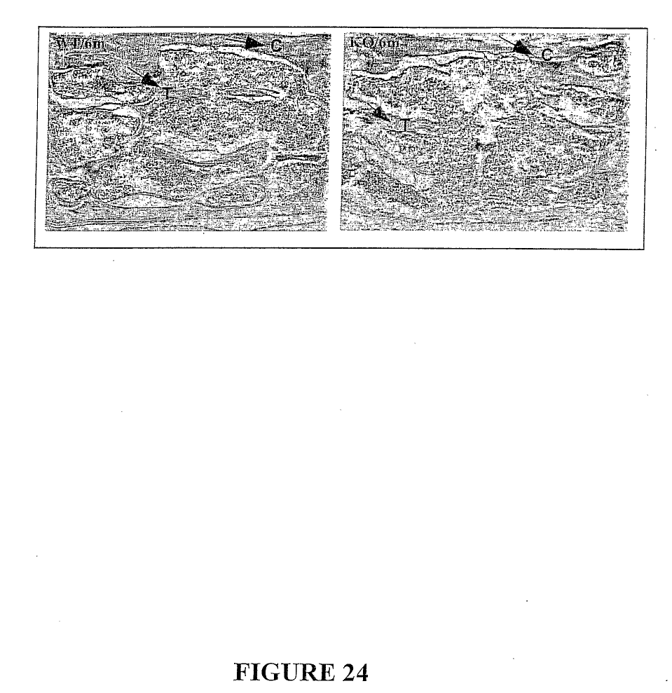

Although 2-3 month old TR4^{-/-} mice showed no detectable skeletal abnormalities and had similar bone mineral density (BMD) compared to aged matched TR4^{+/+} mice, radiographs of 6 month old TR4^{-/-} mice revealed a severe kyphosis (curvature of the spinal column) (FIG. 3a), a landmark for aging bone. DEXA scanning showed a significant global BMD reduction in 6-7 month old TR4^{-/-} mice with increased reduction of BMD in TR4^{-/-} tails representing of spinal (FIG. 13), but there is no change of BMD in the skull between TR4^{-/-} and TR4^{+/+} was similar. Histological analysis of cross-sections from 6 month old TR4^{-/-} thoracic spine displayed a significant reduction on cortical and trabecular bone area compared with TR4^{+/+} mice, which further confirmed osteoporosis phenotypes in 6 month old TR4^{-/-} mice (FIG. 24). It was observed that the reduction of BMD in TR4^{-/-} mice was found in both male and female over age 6 month old, indicating that TR4^{-/-} mice develop a senile osteoporosis which is similar to the type II osteoporosis found in aged human.

TR4^{-/-} female mice are subfertile. Over time, TR4^{-/-} mice appear to become gradually infertile. As shown in FIG. 4a, at the age of 3 months of TR4^{-/-} mice, there are no obvious abnormalities in the gross look of female reproductive organs as compared to aged-matched TR4^{+/+} mice. However, 6 month old TR4^{-/-} mice display premature ovarian dysfunction in which hypotrophy were observed in the reproductive organs and no active estrus cycle could be detected. Histological analysis of ovaries found that TR4^{-/-} ovaries were very small and contained immature preantral and small antral follicles, but no preovulatory follicles. In addition, little interstitum and absent corpora lutea implied complete anovulation (FIG. 4b). All of these findings indicated an early loss of female fertility in TR4^{-/-}.

b) Elevation of ROS Status and Reduced ROS Tolerance in Tr4^{-/-} MEFs Contribute to Rapid Replicative Senescence in Tr4^{-/-} MEFs.

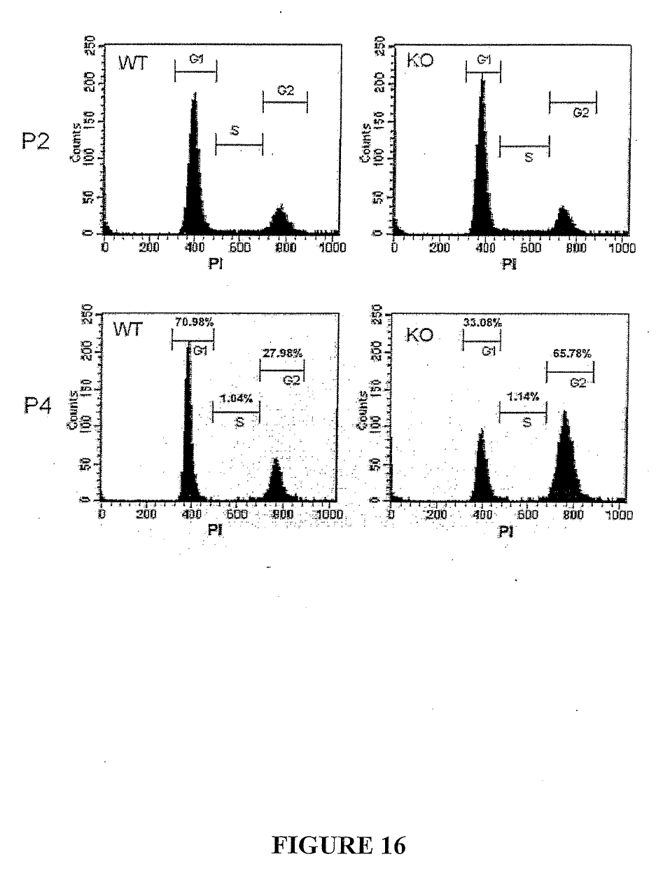

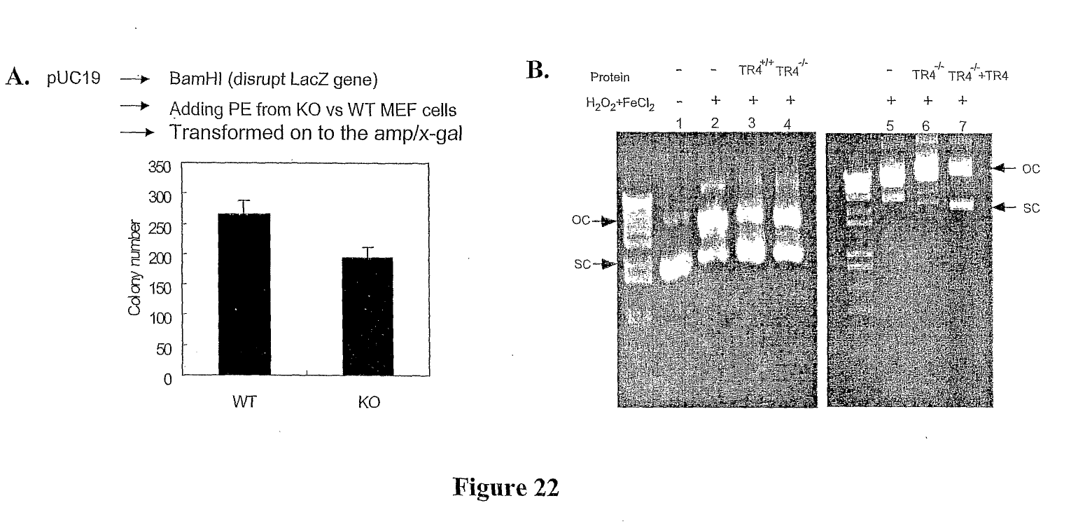

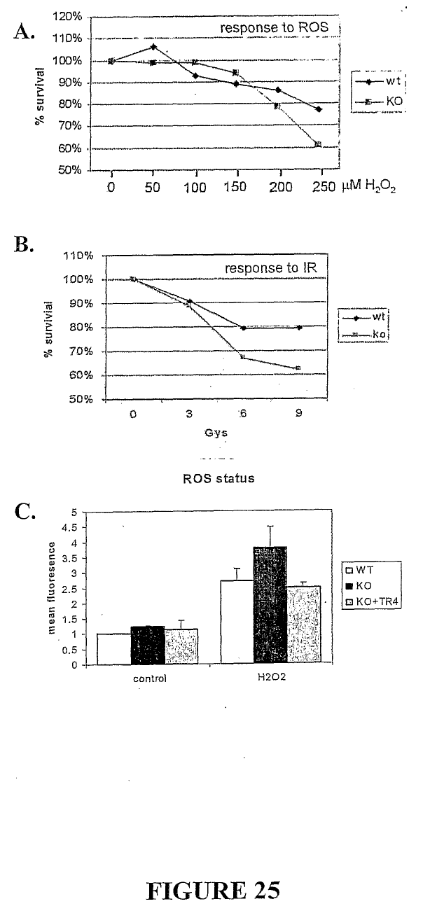

Replicative senescence has been employed as a cellular model for aging; many mutations in DNA repair genes that cause premature aging phenotypes also confer premature replicative senescence in MEFs. Senescence of normal MEFs occurs primarily in response to oxidative DNA damage incurred during cell culture in a process termed extrinsic senescence. As expected, MEFs from TR4^{+/+} mice grow well for approximately 2-3 population doublings (P2-3) before proliferation begins to decline, and after 8-10 doublings, the cell became senescent. In contrast, TR4^{-/-} MEFs display similar cell cycle profiles in the early generations (P1-3), and then their proliferation declined drastically after P4. As shown in FIG. 16, cell cycle profiles were similar in early generations of MEFs from both TR4^{+/+} and TR4^{-/-}. However a G2/M growth arrest was detected in TR4^{-/-} MEFs after four generations (P4), while TR4^{+/+} MEFs retain normal cell cycle profile distribution. ROS is a source of chronic and persistent DNA damage in all cells and contribute to cell senescence and a reduced ability to tolerate stresses would lead to aging. Therefore, how TR4^{+/+} and TR4^{-/-} MEFs respond to stresses was examined. Indeed, it was found that TR4^{-/-} MEFs were more susceptible to stress induced by both H₂O₂ and ionizing radiation (IR) in which fewer TR4^{-/-} MEFs survive than TR4^{+/+} MEFs in higher doses of ROS and IR-induced stress conditions (FIGS. 25A and 25B). To further test how TR4 protects cells from ROS-induced cell senescence, the amount of intracellular ROS in MEFs was examined by flow cytometry using define DCFH-DA. As shown in FIG. 25C, cellular ROS level was higher in TR4^{-/-} than in TR4^{+/+} MEFs. When MEFs were exposed to H₂O₂, ROS levels in both in TR4^{+/+} and TR4^{-/-} were increased; however TR4^{-/-} MEFs accumulated more intracellular ROS than TR4^{+/+} MEFs. Importantly, restoring TR4 into TR4^{-/-} MEFs reduced both endogenous and exogenously H₂O₂-induced ROS generation significantly. To test if TR4 was involved in ROS scavenging system, the protective effects of pUC19 DNA breaks induced by hydroxyl radicals on cellular proteins from TR4^{+/+} and TR4^{-/-} MEFs was measured. As shown in FIG. 22B, treatment with Fe²⁺-H₂O₂ causes plasmid DNA breaks and releasing supercoiled (SC) forms (lane 1) into open circular (OC) forms (lanes 2, and 5); protective effects against DNA breaks from SC into OC forms were found in the presence of TR4^{+/+} MEF protein extracts (lane 3). In contrast, there was less protective effect in the presence of TR4^{-/-} MEF protein extracts which OC forms were present mainly (lanes 4, and 6). Restoring TR4 into TR4^{-/-} reduced the hydroxyl radical-induced DNA breaks, resulting in more DNA break protective effects (lane 6 vs lane 7). These data indicate that TR4 promotes the anti-ROS defense capacity via mediating ROS-scavenging pathways.

Major sources of cellular ROS production come from mitochondria, which decline as a function of age. As shown in FIG. 2, using trichrome staining, abnormal mitochondria accumulation was found in TR4^{-/-} mice muscle in which red-ragged fibers were seen 6 month TR4^{-/-} soleus muscle, in which is absence in TR4^{+/+} muscle fibers. Electron microscopy further confirmed the abnormal, enlargement of mitochondria accumulation in TR4^{-/-} muscle. These larger mitochondria, which can be found in aged mammals, are often not as bio-energetically efficient as the youthful, normal, small mitochondria.

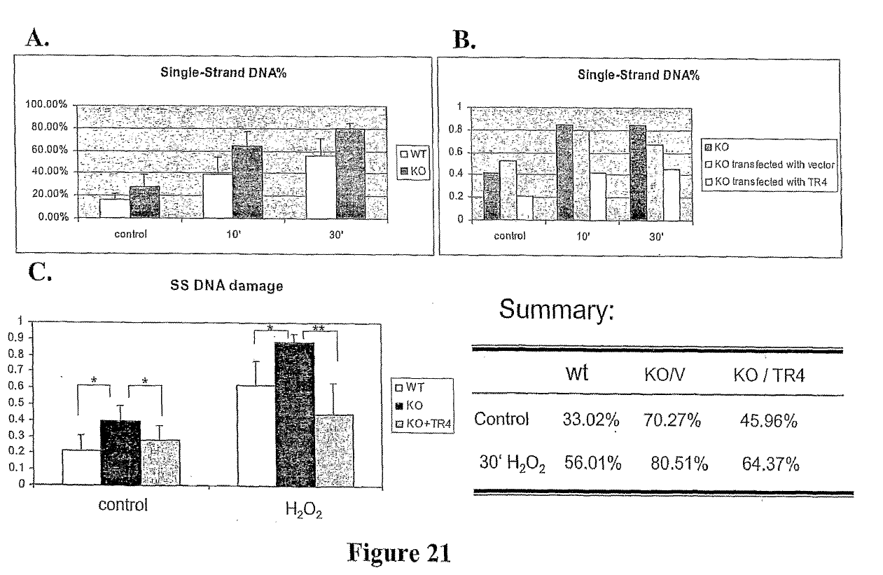

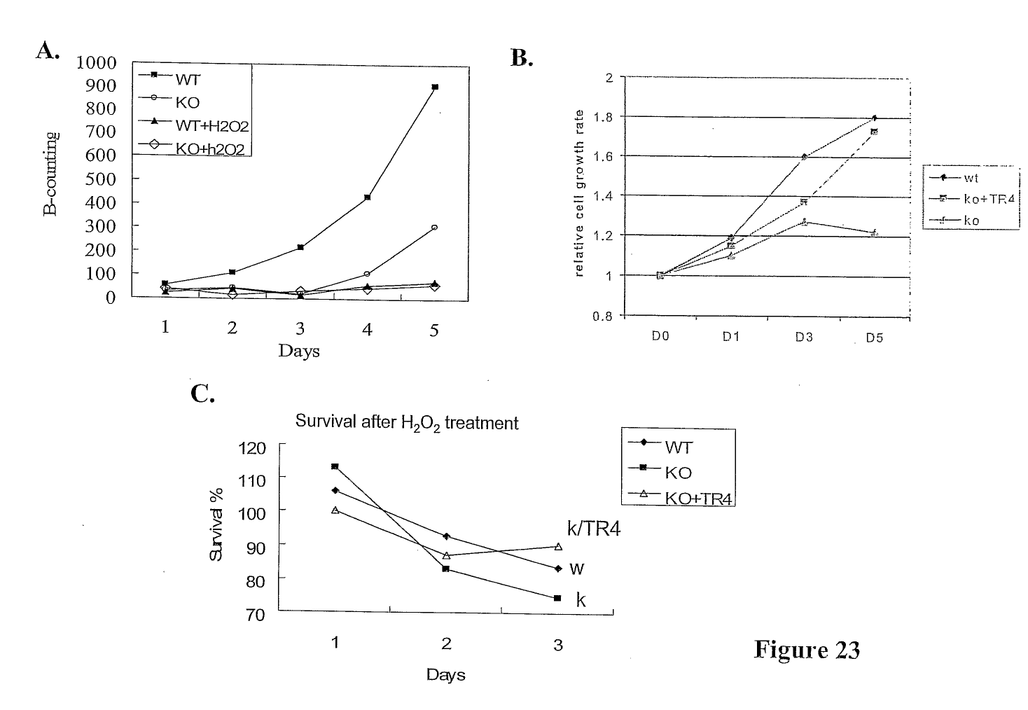

Increased single strand DNA breaks in TR4^{-/-} MEF cells. To test if elevated ROS in TR4^{-/-} MEF would result in more DNA damage, single strand (SS) DNA breaks were measured in MEFs before and after H₂O₂ treatment. As shown in FIG. 21C, the intrinsic as well as extrinsic (H₂O₂-induced) SS DNA breaks increased in TR4^{-/-} MEFs compare to TR4^{+/+} MEFs and restoring TR4 via pBabe retrovirus infection into TR4^{-/-} MEFs reduced SS DNA breaks. DNA breaks without proper and prompted DNA repair lead to genomic instability, and then eventually lead to cellular decay and cell growth arrest. The growth rates were then compared between TR4^{+/+} and TR4^{-/-} MEFs at p3 by MTT assay. Results revealed that TR4^{+/+} MEFs grow faster than TR4^{-/-} and restoring TR4 into TR4/MEFs (TR4^{-/-}+TR4) indeed can slow down the growth retardation in TR4^{-/-} MEFs (FIG. 23B).

Together, the results indicate that higher production of intracellular ROS, less resistance to oxidative stress, and much more severe DNA damage in TR4^{-/-} than TR4^{+/+}, contribute to an early onset of TR4^{-/-} MEFs cellular senescence, and premature aging in TR4^{-/-} mice. Therefore, TR4 might function through antioxidant augmentation to eliminate ROS and/or through participating in DNA repair systems to maintain the genomic integrity.

c) TR4 might be involved in Gadd45α-Mediated DNA Damage Stress-Induced DNA Repair Pathway.

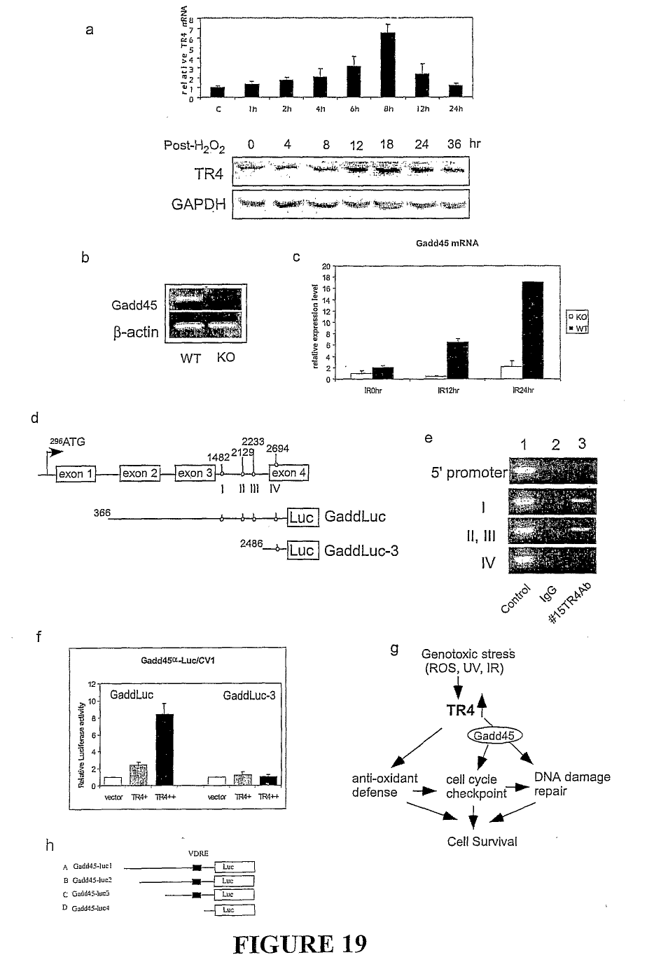

To dissect potential molecular mechanism by which TR4 is involved in the ROS/DNA damage/DNA repair signal cascades, TR4 expression levels were examined upon exposure to genotoxic stress (such as H₂O₂). As shown in FIG. 19a, TR4 mRNA and protein expressions were increased upon H₂O₂ treatment at 4 hr and 8 hr individually, in C2C12 cells. Similar results were observed when the cells were treated with UV and gamma-irradiation, indicating that TR4 is involved, not exclusively, in either some checkpoint proteins that monitor cell-cycle progression, or to translate these DNA-derived stimuli to biochemical signals. In an effort to find molecule(s)/signal(s) that are mediated by TR4 and responsible for age-related defects, it was observed that Gadd45 cc, a growth arrest and DNA damage response gene, appears to be a direct target of TR4. A reduction of Gadd45α mRNA was seen in TR4^{-/-} mice muscle as well as liver at 6 month old mice as compared to TR4^{+/+} mice (FIG. 19b). Gadd45α mRNA expression was not increased upon ionic irradiation in TR4^{-/-} MEFs while Gadd45a mRNA was induced in TR4^{+/+} MEFs 12 hrs after exposed to 6 Gys IR (FIG. 19c). As shown in FIG. 19d, several direct repeat 3 (DR3)-like nuclear receptor response elements have been identified in the third intron of Gadd45a leading us to hypothesize that TR4 can bind to DNA elements that contain DR3-like sequences and transcriptionally regulate Gadd45α gene expression. Using chromosome co-immunoprecipitation assays, TR4 was found to directly bind to DR3-like sequences in the regions I, II, and III, but not region IV of Gadd45 intron 3 (FIG. 19e). Reporter gene assays further showed that TR4 activated Gadd45Luc activity in a TR4-dose dependent manner, but not the luciferase reporter containing only region IV, (GaddLuc3) (FIG. 19f). These findings indicate that TR4 regulates the cell response to stresses by inducing DNA repair thereby affecting the organism life span, and Gadd45α can be one of the effectors modulated by TR4 (FIG. 19g).

d) Discussion

Aging occurs as a consequence of imperfect genomic maintenance. Genomic integrity and cell proliferation/survival are regulated by an intricate network of pathways that include anti-oxidant defense systems, DNA repair, and cell-cycle checkpoints. Consistent with the free-radical theory of aging, TR4^{-/-} MEFs have higher intracellular ROS levels as well as single strand DNA break, both of which support that DNA lesions caused by oxidative damage would ultimately lead to premature aging. Therefore, reducing oxidative stress remains a key in the goal of delaying aging or aging-associated disease. Expression of TR4 in TR4^{-/-} MEFs reduced the endogenous ROS levels, defended exogenous H₂O₂ insults as well as slowed the cellular decay, indicating that TR4 is a potent anti-ROS molecule. The fact that TR4 expression levels can be stimulated upon various genotoxic stresses also indicated TR4 roles in DNA-damage surveillance signal networks that sense the DNA damages and corrects them, if errors occurs. During the course of evolution, numerous genes coding for proteins involved in the surveillance and restoration of DNA integrity have evolved. As a member of the steroid hormone receptor superfamily, TR4 is the first gene in this superfamily involved directly in maintaining the genome integrity.

Reduced DNA repair capacity caused by mutations in DNA repair genes is often linked to developmental abnormalities, neurological disorders, and premature aging syndromes. Several lines of evidence indicate that TR4^{-/-} mice might also suffer from developmental defects. First, lower number of than expected TR4^{-/-} pups/litter were generated from heterozygous pairings indicating an increased embryologic mortality and a possible role of TR4 in the developmental embryonic stage. Second, higher motility rate in young TR4^{-/-} mice and surviving TR4^{-/-} mice reveal developmental abnormalities, including growth retardation, and neurological disorders, finally those surviving TR4^{-/-} mice develop age-related syndromes in their early mid-life age. Those abnormalities found in TR4^{-/-} mice represent an accelerated physiological decay processing with pathological changes induced by the DNA damage-induced genome dysfunction.

Similar to Gadd45-null mice which exhibit severe genomic instabilities and are susceptible to DNA-damage induced tumors, inadequate DNA repair capacity via down-regulation of the Gadd45α in TR4^{-/-} leads to persistent DNA damages and genome instability and eventually results in premature aging. Conclusions from the current studies indicate that TR4 has characteristics as a caretaker tumor suppressor gene which is directly involved in the DNA-damage-repair defense systems to maintain the genome stability.

e) Methods:

(1) Generation and Genotyping of Tr4^{-/-} Mice.

TR4^{-/-} mice were produced as described (X), and housed in vivarium facility of the University of Rochester Medical Center. The animals were provided a standard diet with constant access to food and water, and exposed to a 12-hour light/dark cycle. All experiments protocols were approved by the University Committee on Animal Resources and the office of Environmental Health and Safety. Genotyping was carried out as described. Briefly, genomic DNA was isolated from mouse tail samples and used as template for PCR.

(2) Tissue Preparation and Histology.

Mice were anesthetized with an overdose of pentobarbital and tissues were removed and fixed by submission in 10% neutral buffered formalin. Tissues were cut into 5 □m sections, deparaffinized and stained with haematoxylin and eosin by standard procedure.

(3) Bone Analyses.

Mice were X-rayed in situ under anesthesia for whole body X-ray. Bone mineral density was quantified by DEXA scanning.

(4) Generation of MEF Cells.

Heads and all the internal organs from E14.5 embryos, and then rinsed remanding with PBS, added 5 ml of DMEM medium then passed through a 22 gauge needle few times to mince tissues. Allow the fibroblasts to attach to the culture flask for 25 h, and change the medium to remove unattached cells and debris. MEFs, at passage 0 (P0) would form a confluent monolayer after 2-3 days. Cells were then trypsinized, and sub-cultured for genotyping and experiments. All the experiments were finished before P4.

(5) Protective Effect of Cellular Protein on pUC19 DNA Damage

To determine the protective effects of cellular proteins on plasmid DNA, 5 μl 0.25 μg/μl pUC19 DNA was incubated with 5 μl cellular proteins from TR4^{+/+} or TR4^{-/-} MEFs (PBS was used as control). Then 1 μl 6 mmol/L H₂O₂, and 1 μl 0.4 mmol/L FeSO₄ were added and incubated at 37° C. for 60 min. The reaction was electrophoresed on an agarose gel and DNA damage evaluations were based on the loss of supercoiled (SC) monomer.

(6) Cell Cycle Profiling.

MEFs from TR4^{+/+} or TR4^{-/-} were collected and fixed with 70-75% EtOH at 4° C. for at least 12 h. Cells were then centrifuged at 1000 rpm for 7 min at 4° C. and supernatant decanted, and add 1 ml RNase (1 mg/ml in 1×PBS) for 30 min. Cells were then incubated with 500-1000 μl propidium iodide (20 μg/ml) and analyzed by flow cytometry.

(7) Growth Assay (MTT).

Seed 2000 cells in 96-well plate, and wait at least 12 h or overnight for attachment. Infect retrovirus (vector and TR4 virus) for 24 h. Wash with culture medium and replace with 200 uM H₂O₂ containing medium for 2 hr. Cells without H₂O₂ treatment are Day 0. After 2 hr H₂O₂ treatment, wash cells with culture medium and replace the fresh medium. Harvest cells at day 1, day 3, and day 5 for MTT assay. To determine the cells sensitivity to stress, MEFs from TR4^{-/-} and TR4^{+/+} were seeded, and treated with indicated dose gamma irradiation, and 2 h of H₂O₂. Harvest the cells at day 3 for determining cell growth by MTT assay. The survival rate was determined as the ratio between treated- and non-treated groups.

(8) Measurement of DNA Single-Strand Breaks.

A DNA precipitation assay was used for DNA-strand-breaks detection. Confluent MEFs from TR4^{-/-} and TR4^{+/+} were labeled with 0.25 μCi/ml [³H]methylthymidine for 24 h, then the cells were thoroughly washed with PBS, and supplied with serum free medium in the presence and absence of 250 μM H₂O₂ for another 30 mins. The cells were washed and lysed with lysis buffer (10 nM Tris/HCl/10 mM EDTA/50 mM NaOH/2% SDS, pH 12.4) followed by addition of KCl (12 mM) for 10 min at 65° C., followed by a 5 min cooling-and-precipitation period on ice. A DNA-protein K-SDS precipitate was formed under these conditions, from which low-molecular-mass broken DNA was released. DNA fragments were recovered in the supernatant from a 10 min centrifugation at 200 g, at 10° C., and transferred into a liquid scintillation vial containing 1 ml of 50 mM HCl. The precipitated pellet (intact double-stranded DNA) was solubilized with water at 65° C. Radioactivity was determined by scintillation counter. The amount of double-stranded DNA remaining was calculated for each sample by dividing the d.p.m. value of the pellet by the total d.p.m. value of the pellet plus supernatant and multiplying by 100. The results representing the extent of DNA damage were calculated as (Dt/Dc)×100, where Dt represents double-stranded DNA in treated cells and Dc represents double-stranded DNA in the respective control cells. In control cells (cells incubated in Ca²⁺-containing or Ca²⁺-free/EGTA), the level of total double-stranded DNA was around 75%. Pretreatment with the various chelators did not affect this level.

(9) Measurement of Intracellular ROS by Flow Cytometry.

Production of intracellular ROS was detected by flow cytometry using DCFH-DA. The MEFs from TR4^{-/-} and TR4^{+/+} were cultured in 35-mm tissue-culture dishes. The cells were treated with 250 μM H₂O₂, when they reached 80% confluent, cells were for 30 min in serum free medium, cells were incubated with 10 μM DCFH-DA for 30 min under 37° C. degree in the dark, washed once with PBS, detached by trypsinization, collected by centrifugation, and suspended in PBS prior to flow cytometry. For the control group, the medium was changed to serum free medium and incubated with 10 μM DCFH and followed by flow cytometric analyses, and the amount of cellular ROS levels was quantified by the fluorescence of dichlorofluorescein (DCF) density.

(10) Chromatin Immunoprecipitation (ChIP).

PCMX-TR4 transfected H1299 cells were fixed by addition of formaldehyde (1%) and stopped with glycine (0.125 M). The cells were then washed with phosphate-buffered saline, and lysed with lysis buffer (5 mM Pipes/pH 8.0, 85 mM KCl, and 0.5% NP-40 with protease inhibitor). The nuclei were collected by centrifugation and resuspended in nuclear lysis buffer (50 mM Tris/pH 8.1, 10 mM EDTA, and 1% SDS with protease inhibitor). The samples were sonicated and microcentrifuged. The chromatin solution was precleared with protein A/G-Sepharose (Pierce) for 30 min at 4° C. 1 μg anti-TR4 (N15) was added, and incubated overnight. Immunoprecipitations (IPs) complexes were incubated with 45 μl protein A/G-Sepharose and 2 μg salmon sperm DNA for 1 h at 4° C. with rotation. Precipitates were washed sequentially for 3-5 min each in low salt buffer (0.1% SDS, 1% Triton X-100, 2 mM EDTA, 20 mM Tris-HCl/pH 8.1, 150 mM NaCl), high salt buffer (0.1% SDS, 1% Triton X-100, 2 mM EDTA, 20 mM Tris-HCl/pH 8.1, 500 mM NaCl), LiCl wash buffer (0.25 M LiCl, 1% NP-40, 1% deoxycholate, 1 mM EDTA, 10 mM Tris-HCl/pH 8.1), and twice in 1×TE buffer. Precipitates were extracted three times with 1% SDS and 0.1M NaHCO₃. Elutes were then pooled and incubated at 65° C. water bath for 4-5 h to reverse the formaldehyde cross-linking. DNA fragments were purified with a QIAquick Spin Kit (Qiagen), and resuspended in 30 μl TE (47, 48). TR4 antibody pull-down DNA fragments were tested for their association with four regions located in Gadd45α intron 3, and exon 4 that contain DR3 motif. The sequences are described as following:

| | |
|---|---|
| region I (sense: | |
| 5′-GGTTGCCTGATTGTGGATCTGTG-3′, | (SEQ ID NO: 25) |
| antisense: | |
| 5′-GCTGACTCCTTAATGAGGGGTGAG-3′, | (SEQ ID NO: 26)) |
| region II, and III (sense: | |
| 5′-ACAGCCCGATTATTTTGCTACTCC-3′, | (SEQ ID NO: 27) |
| antisense: | |
| 5′-TTTCTTCAAGGTAGTTGGGTTCCC-3′, | (SEQ ID NO: 28)) |
| region IV (sense: | |
| 5′-TGAACGGTGATGGCATCTGAATG-3′, | (SEQ ID NO: 29) |
| antisense: | |
| 5′-TTTTCCTTCCTGCATGGTTCTTTG-3′ | (SEQ ID NO: 30)) |
| and | |
| promoter: (sense: | |
| 5′-TGTGTGGGTGTCAGATGGTTGTC-3′; | (SEQ ID NO: 31) |
| antisense: | |
| 5′-TTATTTCGGTGCCCTGATGG-3′. | (SEQ ID NO: 32)) |

### REFERENCES

Ames, B. N. Delaying the mitochondrial decay of aging. Ann N Y Acad Sci, 1019: 406-411, 2004.
Ames, B. N. Mitochondrial decay, a major cause of aging, can be delayed. J Alzheimers Dis, 6: 117-121, 2004.
Barrou, Z., Charru, P., and Lidy, C. Dehydroepiandrosterone (DHEA) and aging. Arch Gerontol Geriatr, 24: 233-241, 1997.
Beato, M. and Klug, J. Steroid hormone receptors: an update. Hum Reprod Update, 6: 225-236, 2000.
Beato, M. Transcriptional control by nuclear receptors. Faseb J, 5: 2044-2051, 1991.
Bertoni-Freddari, C., Fattoretti, P., Casoli, T., Spagna, C., and Meier-Ruge, W. Morphological alterations of synaptic mitochondria during aging. The effect of Hydergine treatment. Ann NY Acad Sci, 717: 137-149, 1994.
Bohr, V. A., Brosh, R. M., Jr., von Kobbe, C., Opresko, P., and Karmakar, P. Pathways defective in the human premature aging disease Werner syndrome. Biogerontology, 3: 89-94, 2002.
Bohr, V. A., Stevnsner, T., and de Souza-Pinto, N.C. Mitochondrial DNA repair of oxidative damage in mammalian cells. Gene, 286: 127-134, 2002.
Brown, W. T. Genetic diseases of premature aging as models of senescence. Annu Rev Gerontol Geriatr, 10: 23-42, 1990.
Brown, W. T. Premature aging syndromes. Curr Probl Dermatol, 17: 152-165, 1987.
Chang, C., Da Silva, S. L., Ideta, R., Lee, Y., Yeh, S., and Burbach, J. P. Human and rat TR4 orphan receptors specify a subclass of the steroid receptor superfamily. Proc Natl Acad Sci USA, 91: 6040-6044, 1994.
Collins, L. L., Lee, Y. F., Heinlein, C. A., Liu, N.C., Chen, Y. T., Shyr, C. R., Meshul, C. K., Uno, H., Plaft, K. A., and Chang, C. Growth retardation and abnormal maternal behavior in mice lacking testicular orphan nuclear receptor 4. Proc Natl Acad Sci U S A, 101: 15058-15063, 2004.
da-Silva, W. S., Gomez-Puyou, A., de Gomez-Puyou, M. T., Moreno-Sanchez, R., De Felice, F. G., de Meis, L., Oliveira, M. F., and Galina, A. Mitochondrial bound hexokinase activity as a preventive antioxidant defense: steady-state ADP formation as a regulatory mechanism of membrane potential and reactive oxygen species generation in mitochondria. J Biol Chem, 279: 39846-39855, 2004.
Davis, T., Singhrao, S. K., Wyllie, F. S., Haughton, M. F., Smith, P. J., Wiltshire, M., Wynford-Thomas, D., Jones, C. J., Faragher, R. G., and Kipling, D. Telomere-based proliferative lifespan barriers in Werner-syndrome fibroblasts involve both p53-dependent and p53-independent mechanisms. J Cell Sci, 116: 1349-1357, 2003.
de Boer, J., Andressoo, J. O., de Wit, J., Huijmans, J., Beems, R. B., van Steeg, H., Weeda, G., van der Horst, G. T., van Leeuwen, W., Themmen, A. P., Meradji, M., and Hoeijmakers, J. H. Premature aging in mice deficient in DNA repair and transcription. Science, 296: 1276-1279, 2002.
Djordjevic-Markovic, R., Radic, O., Jelic, V., Radojcic, M., Rapic-Otrin, V., Ruzdijic, S., Krstic-Demonacos, M., Kanazir, S., and Kanazir, D. Glucocorticoid receptors in ageing rats. Exp Gerontol, 34: 971-982, 1999.
Droge, W. Oxidative stress and aging. Adv Exp Med Biol, 543: 191-200, 2003.
Genova, M. L., Pich, M. M., Bemacchia, A., Bianchi, C., Biondi, A., Bovina, C., Falasca, A. I., Formiggini, G., Castelli, G. P., and Lenaz, G. The mitochondrial production of reactive oxygen species in relation to aging and pathology. Ann N Y Acad Sci, 1011: 86-100, 2004.

Hasty, P., Campisi, J., Hoeijmakers, J., van Steeg, H., and Vijg, J. Aging and genome maintenance: lessons from the mouse? Science, 299: 1355-1359, 2003.
Hsieh, T. J., Liu, T. Z., Chia, Y. C., Chem, C. L., Lu, F. J., Chuang, M. C., Mau, S. Y., Chen, S. H., Syu, Y. H., and Chen, C. H. Protective effect of methyl gallate from Toona sinensis (Meliaceae) against hydrogen peroxide-induced oxidative stress and DNA damage in MDCK cells. Food Chem Toxicol, 42: 843-850, 2004.
Huang, H. and Manton, K. G. The role of oxidative damage in mitochondria during aging: a review. Front Biosci, 9: 1100-1117, 2004.
Jacobs, H. T. The mitochondrial theory of aging: dead or alive? Aging Cell, 2: 11-17, 2003.
Jornot, L., Petersen, H., and Junod, A. F. Hydrogen peroxide-induced DNA damage is independent of nuclear calcium but dependent on redox-active ions. Biochem J, 335 (Pt 1): 85-94, 1998.
Kim, E., Xie, S., Yeh, S. D., Lee, Y. F., Collins, L. L., Hu, Y. C., Shyr, C. R., Mu, X. M., Liu, N.C., Chen, Y. T., Wang, P. H., and Chang, C. Disruption of TR4 orphan nuclear receptor reduces the expression of liver apolipoprotein E/C-1/C-II gene cluster. J Biol Chem, 278: 46919-46926, 2003.
Kulesza, P. and Lieber, M. R. DNA-PK is essential only for coding joint formation in V(D)J recombination. Nucleic Acids Res, 26: 3944-3948, 1998.
Kuro-o, M., Matsumura, Y., Aizawa, H., Kawaguchi, H., Suga, T., Utsugi, T., Ohyama, Y., Kurabayashi, M., Kaname, T., Kume, E., Iwasaki, H., Iida, A., Shiraki-Iida, T., Nishikawa, S., Nagai, R., and Nabeshima, Y. I. Mutation of the mouse klotho gene leads to a syndrome resembling ageing. Nature, 390: 45-51, 1997.
Lee, Y. F., Lee, H. J., and Chang, C. Recent advances in the TR2 and TR4 orphan receptors of the nuclear receptor superfamily. J Steroid Biochem Mol Biol, 81: 291-308, 2002.
Lee, Y. F., Shyr, C. R., Thin, T. H., Lin, W. J., and Chang, C. Convergence of two repressors through heterodimer formation of androgen receptor and testicular orphan receptor-4: a unique signaling pathway in the steroid receptor superfamily. Proc Natl Acad Sci USA, 96: 14724-14729, 1999.
Lee, Y. F., Young, W. J., Burbach, J. P., and Chang, C. Negative feedback control of the retinoid-retinoic acid/retinoid X receptor pathway by the human TR4 orphan receptor, a member of the steroid receptor superfamily. J Biol Chem, 273: 13437-13443, 1998.
Lee, Y. F., Young, W. J., Lin, W. J., Shyr, C. R., and Chang, C. Differential regulation of direct repeat 3 vitamin D3 and direct repeat 4 thyroid hormone signaling pathways by the human TR4 orphan receptor. J Biol Chem, 274: 16198-16205, 1999.
Lehmann, A. Ageing: repair and transcription keep us from premature ageing. Curr Biol, 12: R550-551, 2002.
Liu, J., Atamna, H., Kuratsune, H., and Ames, B. N. Delaying brain mitochondrial decay and aging with mitochondrial antioxidants and metabolites. Ann N Y Acad Sci, 959: 133-166, 2002.
Mandavilli, B. S., Santos, J. H., and Van Houten, B. Mitochondrial DNA repair and aging. Mutat Res, 509: 127-151, 2002.
Martin, G. M. and Loeb, L. A. Ageing: mice and mitochondria. Nature, 429: 357-359, 2004.
Martin, G. M. Syndromes of accelerated aging. Natl Cancer Inst Monogr, 60: 241-247, 1982.
Menzies, F. M., Cookson, M. R., Taylor, R. W., Turnbull, D. M., Chrzanowska-Lightowlers, Z. M., Dong, L., Figlewicz, D. A., and Shaw, P. J. Mitochondrial dysfunction in a cell culture model of familial amyotrophic lateral sclerosis. Brain, 125: 1522-1533, 2002.
Miquel, J. An update on the oxygen stress-mitochondrial mutation theory of aging: genetic and evolutionary implications. Exp Gerontol, 33: 113-126, 1998.
Mu, X., Lee, Y. F., Liu, N.C., Chen, Y. T., Kim, E., Shyr, C. R., and Chang, C. Targeted inactivation of testicular nuclear orphan receptor 4 delays and disrupts late meiotic prophase and subsequent meiotic divisions of spermatogenesis. Mol Cell Biol, 24: 5887-5899, 2004.
Nyaga, S. G. and Bohr, V. A. Characterization of specialized mtDNA glycosylases. Methods Mol Biol, 197: 227-244, 2002.
Parrinello, S., Samper, E., Krtolica, A., Goldstein, J., Melov, S., and Campisi, J. Oxygen sensitivity severely limits the replicative lifespan of murine fibroblasts. Nat Cell Biol, 5: 741-747, 2003.
Roubertoux, P. L., Sluyter, F., Carlier, M., Marcet, B., Maarouf-Veray, F., Cherif, C., Marican, C., Arrechi, P., Godin, F., Jamon, M., Verrier, B., and Cohen-Salmon, C. Mitochondrial DNA modifies cognition in interaction with the nuclear genome and age in mice. Nat Genet, 35: 65-69, 2003.
Shyr, C. R., Hu, Y. C., Kim, E., and Chang, C. Modulation of estrogen receptor-mediated transactivation by orphan receptor TR4 in MCF-7 cells. J Biol Chem, 277: 14622-14628, 2002.
Stevnsner, T., Thorslund, T., de Souza-Pinto, N.C., and Bohr, V. A. Mitochondrial repair of 8-oxoguanine and changes with aging. Exp Gerontol, 37: 1189-1196, 2002.
Suliman, H. B., Carraway, M. S., Velsor, L. W., Day, B. J., Ghio, A. J., and Piantadosi, C. A. Rapid mtDNA deletion by oxidants in rat liver mitochondria after hemin exposure. Free Radic Biol Med, 32: 246-256, 2002.
Sun, L. Q., Lee, D. W., Zhang, Q., Xiao, W., Raabe, E. H., Meeker, A., Miao, D., Huso, D. L., and Arceci, R. J. Growth retardation and premature aging phenotypes in mice with disruption of the SNF2-like gene, PASG. Genes Dev, 18: 1035-1046, 2004.
Tanhauser, S. M. and Laipis, P. J. Multiple deletions are detectable in mitochondrial DNA of aging mice. J Biol Chem, 270: 24769-24775, 1995.
Taylor, R. W., Chinnery, P. F., Turnbull, D. M., and Lightowlers, R. N. Selective inhibition of mutant human mitochondrial DNA replication in vitro by peptide nucleic acids. Nat Genet, 15: 212-215, 1997.
Tian, B., Wu, Y., Sheng, D., Zheng, Z., Gao, G., and Hua, Y. Chemiluminescence assay for reactive oxygen species scavenging activities and inhibition on oxidative damage of DNA in Deinococcus radiodurans. Luminescence, 19: 78-84, 2004.
Tieu, K., Perier, C., Caspersen, C., Teismann, P., Wu, D.C., Yan, S. D., Naini, A., Vila, M., Jackson-Lewis, V., Ramasamy, R., and Przedborski, S. D-beta-hydroxybutyrate rescues mitochondrial respiration and mitigates features of Parkinson disease. J Clin Invest, 112: 892-901, 2003.
Tran, H., Brunet, A., Grenier, J. M., Datta, S. R., Fornace, A. J., Jr., DiStefano, P. S., Chiang, L. W., and Greenberg, M. E. DNA repair pathway stimulated by the forkhead transcription factor FOXO3a through the Gadd45 protein. Science, 296: 530-534, 2002.
Trifunovic, A., Wredenberg, A., Falkenberg, M., Spelbrink, J. N., Rovio, A. T., Bruder, C. E., Bohlooly, Y. M., Gidlof, S., Oldfors, A., Wibom, R., Tomell, J., Jacobs, H. T., and Larsson, N. G. Premature ageing in mice expressing defective mitochondrial DNA polymerase. Nature, 429: 417-423, 2004.
Tyner, S. D., Venkatachalam, S., Choi, J., Jones, S., Ghebranious, N., Igelmann, H., Lu, X., Soron, G., Cooper, B., Brayton, C., Hee Park, S., Thompson, T., Karsenty, G., Bradley, A., and Donehower, L. A. p53 mutant mice that display early ageing-associated phenotypes. Nature, 415: 45-53, 2002.
Vogel, H., Lim, D. S., Karsenty, G., Finegold, M., and Hasty, P. Deletion of Ku86 causes early onset of senescence in mice. Proc Natl Acad Sci USA, 96:10770-10775, 1999.
Young, W. J., Smith, S. M., and Chang, C. Induction of the intronic enhancer of the human ciliary neurotrophic factor receptor (CNTFRalpha) gene by the TR4 orphan receptor. A member of steroid receptor superfamily. J Biol Chem, 272: 3109-3116, 1997.
Zhang, D., Mott, J. L., Chang, S. W., Denniger, G., Feng, Z., and Zassenhaus, H. P. Construction of transgenic mice with tissue-specific acceleration of mitochondrial DNA mutagenesis. Genomics, 69: 151-161, 2000.
Zhou, X. J., Vaziri, N. D., Wang, X. Q., Silva, F. G., and Laszik, Z. Nitric oxide synthase expression in hypertension induced by inhibition of glutathione synthase. J Pharmacol Exp Ther, 300: 762-767, 2002.

What is claimed is:

1. A method of generating a model for premature aging comprising generating a TR4 knock out animal and assaying the animal for characteristics of aging.

2. A method for testing a compound for an effect on aging, comprising administering the drug to an animal and assaying for TR4 activity, wherein an increase in TR4 activity indicates a compound that can be used to treat the effects of aging.

3. A method of testing a composition for its effect on aging comprising administering the composition to a TR4 knockout animal, and performing an assay related to aging, wherein a change in the assay relative to a control indicates the compound has an effect on aging.

4. A method of testing a subject for premature aging comprising performing an assay for premature aging, wherein the subject has a TR4 deficiency.

5. A method of diagnosing the likelihood of a subject to develop premature aging comprising taking a tissue sample from the subject and assaying for reduced TR4 activity, wherein a decrease in TR4 activity indicates premature aging.

6. A method of diagnosing a subject with premature aging comprising a) obtaining a tissue sample, and b) assaying for TR4 activity, wherein a lack of TR4 indicates premature aging.

7. A method of treating a subject with signs of aging comprising administering to the subject an agent that modulates TR4 activity, wherein an increase in TR4 activity reduces the signs of aging.

8. A method for evaluating whether a treatment with a compound should be performed due to the effect the treatment of a subject has on aging, wherein the compound modulates the TR4 activity, the method comprising exposing cells having TR4 activity to the compound, and evaluating TR4 activity in the presence of the compound, wherein a difference in the TR4 activity, relative to the TR4 activity of the cells that have not been exposed to the compound, indicates that the compound modulates TR4 activity, and wherein a decrease in TR4 activity indicates an adverse effect on aging, providing an indication that treatment with the compound may not be indicated.

9. A method for screening drugs for an effect on aging comprising administering the drug to an animal and assaying for TR4 activity, wherein a increase in TR4 activity indicates a drug that can be used to treat aging.

10. A method of treating a subject with premature aging comprising administering to the subject an agent that modulates TR4 activity, wherein an increase in TR4 activity reduces premature aging.


SEQUENCE LISTING (32 sequences)

SEQ ID NO: 1 - protein, 596 aa, Artificial Sequence
  Description of Artificial Sequence; note=synthetic construct
MTSPSPRIQIISTDSAVASPQRIQIVTDQQTGQKIQIVTAVDASGSPKQQFILTSPDGAGTGKVILASPETSSAKQLIFTTSDNLVPGRIQIVTDSASVERLLGKTDVQRPQVVEYCVVCGDKASGRHYGAVSCEGCKGFFKRSVRKNLTYSCRSNQDCIINKHHRNRCQFCRLKKCLEMGMKMESVQSERKPFDVQREKPSNCAASTEKIYIRKDLRSPLIATPTFVADKDGARQTGLLDPGMLVNIQQPLIREDGTVLLATDSKAETSQGALGTLANVVTSLANLSESLNNGDTSEIQPEDQSASEITRAFDTLAKALNTTDSSSSPSLADGIDTSGGGSIHVISRDQSTPIIEVEGPLLSDTHVTFKLTMPSPMPEYLNVHYICESASRLLFLSMHWARSIPAFQALGQDCNTSLVRACWNELFTLGLAQCAQVMSLSTILAAIVNHLQNSIQEDKLSGDRIKQVMEHIWKLQEFCNSMAKLDIDGYEYAYLKAIVLFSPDHPGLTSTSQIEKFQEKAQMELQDYVQKTYSEDTYRLARILVRLPALRLMSSNITEELFFTGLIGNVSIDSIIPYILKMETAEYNGQITGASL

SEQ ID NO: 2 - protein, 596 aa, Artificial Sequence
  Description of Artificial Sequence; note=synthetic construct
MTSPSPRIQIISTDSAVRSPQRIQIVTDQQTGQKLQIVTAVDASGSSKQQFILTSPDGAGTGKVILASPETSSAKQLIFTTSDNLVPGRIQIVTDSASVERLLGKADVQRPQVVEYCVVCGDKASGRHYGAVSCEGCKGFFKRSVRKNLTYSCRSSQDCIINKHHRNRCQFCRLKKCLEMGMKMESVQSERKPFDVQREKPSNCAASTEKIYIRKDLRSPLIATPTFVADKDGSRQTGLLDPGMLVNIQQPLIREDGTVLLATDSKAETSQGALGTLANVVTSLANLSESLNNGDASEMQPEDQSASEITRAFDTLAKALNTTDSASPPSLADGIDASGGGSIHVISRDQSTPIIEVEGPLLSDTHVTFKLTMPSPMPEYLNVHYICESASRLLFLSMHWARSIPAFQALGQDCNTSLVRACWNELFTLGLAQCAQVMSLSTILAAIVNHLQNSIQEDKLSGDRIKQVMEHIWKLQEFCNSMAKLDIDGHEYAYLKAIVLFSPDHPGLTGTSQIEKFQEKAQMELQDYVQKTYSEDTYRLARILVRLPALRLMSSNITEELFFTGLIGNVSIDSIIPYILKMETAEYNGQITGASL

SEQ ID NO: 3 - protein, 596 aa, Artificial Sequence; identical to SEQ ID NO: 2
  Description of Artificial Sequence; note=synthetic construct

SEQ ID NO: 4 - protein, 615 aa, Artificial Sequence
  Description of Artificial Sequence; note=synthetic construct
MTSPSPRIQIISTDSAVASPQRIQGSEPASGPLSVFTSLNKEKIVTDQQTGQKIQIVTAVDASGSPKQQFILTSPDGAGTGKVILASPETSSAKQLIFTTSDNLVPGRIQIVTDSASVERLLGKTDVQRPQVVEYCVVCGDKASGRHYGAVSCEGCKGFFKRSVRKNLTYSCRSSQDCIINKHHRNRCQFCRLKKCLEMGMKMESVQSERKPFDVQREKPSNCAASTEKIYIRKDLRSPLIATPTFVADKDGARQTGLLDPGMLVNIQQPLIREDGTVLLATDSKAETSQGALGTLANVVTSLANLSESLNNGDTSEIQPEDQSASEITRAFDTLAKALNTTDSSSSPSLADGIDTSGGGSIHVISRDQSTPIIEVEGPLLSDTHVTFKLTMPSPMPEYLNVHYICESASRLLFLSMHRARSIPAFQGLGQDCNTSLVRACWNELFTLGLAQCAQVMSLSTILAAIVNHLQNSIQEDKLSGDRIKQVMEHIWKLQEFCNSMANWDIDGYEYAYLKAIVLFSPDHPGLTSTSQIEKFQEKAQMELQDYVQKTYSEDTYRLARILVRLPALRLMSSNITEELFFTGLIGNVSIDSIIPYILKMETAEYNGQITGVSL

SEQ ID NO: 5 - protein, 615 aa, Artificial Sequence; identical to SEQ ID NO: 4
  Description of Artificial Sequence; note=synthetic construct

SEQ ID NO: 6 - DNA, 2254 bp, Artificial Sequence
  Description of Artificial Sequence; note=synthetic construct
gaattcgccgctcccacctcggcgtctcgtctctcgcccgctgccccgcgagcccgcggcccccggctcccgccatccgccgacaccgggagcccgggctccccgcgccctgccctccgcgccgggggccgcccgccgcagacacgggacccgcttcgaggccgctttggcgcaaatcctgaggtaacacgtacacagacctctcggccggaatctccagggatgaccagcccctccccacgcatccagataatctccaccgactctgctgtagcctcacctcagcgcattcagggctctgaacctgcctctggcccattgagtgttttcacatctttgaacaaagagaagattgtcacagaccagcagacaggacagaaaatccagatagtcaccgcagtggacgcctccggatcccccaaacagcagttcatcctgaccagcccagatggagctggaactgggaaggtgatcctggcttccccagagacatccagcgccaagcaactcatattcaccacctcagacaacctcgtccctggcaggatccagattgtcacggattctgcctctgtggagcgtttactggggaagacggacgtccagcggccccaggtggtagagtactgtgtggtctgtggcgacaaagcctccggccgtcactatggggctgtcagttgtgaaggttgcaaaggtttcttcaaaaggagtgtgaggaaaaatttgacctacagctgccggagcagccaagactgcatcatcaataaacatcaccggaaccgctgtcagttttgccggctgaaaaaatgcttagagatgggcatgaaaatggaatctgtgcagagtgaacggaagcccttcgatgtgcaacgggagaaaccaagcaattgtgctgcttcaactgagaaaatctatatccggaaagacctgagaagtcccctgatagctactcccacgtttgtggcagacaaagatggagcaagacaaacaggtcttcttgatccagggatgcttgtgaacatccagcagcctttgatacgtgaggatggtacagttctcctggccacggattctaaggctgaaacaagccagggagctctgggcacactggcaaatgtagtgacctcccttgccaacctaagtgaatctttgaacaacggtgacacttcagaaatccagccagaggaccagtctgcaagtgagataactcgggcatttgataccttagctaaagcacttaataccacagacagctcctcttctccaagcttggcagatgggatagacaccagtggaggagggagcatccacgtcatcagcagagaccagtcgacacccatcattgaggttgaaggccccctcctttcagacacacacgtcacatttaagctaacaatgcccagtccaatgccagagtacctcaacgtgcactacatctgtgagtctgcatcccgtctgcttttcctctcaatgcaccgggcaaggtcaatcccagcctttcagggacttggacaggactgcaacaccagccttgtgcgggcctgctggaatgagctcttcaccctcggcctggcccagtgtgcccaggtcatgagtctctccaccatcctggctgccattgtcaaccacctgcagaacagcatccaggaagataaactttctggtgaccggataaagcaagtcatggagcacatctggaagctgcaggagttctgtaacagcatggcgaactgggatatagatggctatgagtatgcataccttaaagctatagttctctttagccccgatcatccaggtttgaccagcacaagccagattgaaaaattccaagaaaaggcacagatggagttgcaggactatgttcagaaaacctactcagaagacacctaccgattggcccggatcctcgttcgcctgccggcactcaggctgatgagctccaacataacagaagaacttttttttactggtctcattggcaatgtttcgatagacagcataatcccctacatcctcaagatggagacagcagagtataatggccagatcaccggagtcagtctatagcgcaaaccacacacctgccaaggagcaacagaatccttccaggaccgttcacatacaaagaaaagtagtggtattttggtatgtgcaaatatttccatatgttagccatttcctgtctggtttctccttatctgttaatcccagacaatagcaattaaaagactagtagggatcctttcctgac

SEQ ID NO: 7 - DNA, 2254 bp, Artificial Sequence
  Description of Artificial Sequence; note=synthetic construct
gaattcgccgctcccacctcggcgtctcgtctctcgcccgctgccccgcgagcccgcggcccccggctcccgccatccgccgacaccgggagcccgggctccccgcgccctgccctccgcgccgggggccgcccgccgcagacacgggacccgcttcgaggccgctttggcgcaaatcctgaggtaacacgtacacagacctctcggccggaatctccagggatgactagcccctccccacgcatccagataatctccaccgactctgctgtagcctcacctcagcgcattcagggctctgaacctgcctctggcccattgagtgttttcacatctttgaacaaagagaagattgtcacagaccagcagacaggacagaaaatccagatagtcaccgcagtggacgcctccggatcccccaaacagcagttcatcctgaccagcccagatggagctggaactgggaaggtgatcctggcttccccagagacatccagcgccaagcaactcatattcaccacctcagacaacctcgtccctggcaggatccagattgtcacggattctgcctctgtggagcgtttactggggaagacggacgtccagcggccccaggtggtagagtactgtgtggtctgtggcgacaaagcctccggccgtcactatggggctgtcagttgtgaaggttgcaaaggtttcttcaaaaggagtgtgaggaaaaatttgacctacagctgccggagcagccaagactgcatcatcaataaacatcaccggaaccgctgtcagttttgccggctgaaaaaatgcttagagatgggcatgaaaatggaatctgtgcagagtgaacggaagcccttcgatgtgcaacgggagaaaccaagcaattgtgctgcttcaactgagaaaatctatatccggaaagacctgagaagtcccctgatagctactcccacgtttgtggcagacaaagatggagcaagacaaacaggtcttcttgatccagggatgcttgtgaacatccagcagcctttgatacgtgaggatggtacagttctcctggccacggattctaaggctgaaacaagccagggagctctgggcacactggcaaatgtagtgacctcccttgccaacctaagtgaatctttgaacaacggtgacacttcagaaatccagccagaggaccagtctgcaagtgagataactcgggcatttgataccttagctaaagcacttaataccacagacagctcctcttctccaagcttggcagatgggatagacaccagtggaggagggagcatccacgtcatcagcagagaccagtcgacacccatcattgaggttgaaggccccctcctttcagacacacacgtcacatttaagctaacaatgcccagtccaatgccagagtacctcaacgtgcactacatctgtgagtctgcatcccgtctgcttttcctctcaatgcaccgggcaaggtcaatcccagcctttcagggacttggacaggactgcaacaccagccttgtgcgggcctgctggaatgagctcttcaccctcggcctggcccagtgtgcccaggtcatgagtctctccaccatcctggctgccattgtcaaccacctgcagaacagcatccaggaagataaactttctggtgaccggataaagcaagtcatggagcacatctggaagctgcaggagttctgtaacagcatggcgaactgggatatagatggctatgagtatgcataccttaaagctatagttctctttagccccgatcatccaggtttgaccagcacaagccagattgaaaaattccaagaaaaggcacagatggagttgcaggactatgttcagaaaacctactcagaagacacctaccgattggcccggatcctcgttcgcctgccggcactcaggctgatgagctccaacataacagaagaacttttttttactggtctcattggcaatgtttcgatagacagcataatcccctacatcctcaagatggagacagcagagtataatggccagatcaccggagtcagtctatagcgcaaaccacacacctgccaaggagcaacagaatccttccaggaccgttcacatacaaagaaaagtagtggtattttggtatgtgcaaatatttccatatgttagccatttcctgtctggtttctccttatctgttaatcccagacaatagcaattaaaagactagtagggatcctttcctgac

SEQ ID NO: 8 - protein, 614 aa, Artificial Sequence
  Description of Artificial Sequence; note=synthetic construct
MTSPSPRQIISTDSAVASPQRIQGSEPASGPLSVFTSLNKEKIVTDQQTGQKIQIVTAVDASGSPKQQFILTSPDGAGTGKVILASPETSSAKQLIFTTSDNLVPGRIQIVTDSASVERLLGKTDVQRPQVVEYCVVCGDKASGRHYGAVSCEGCKGFFKRSVRKNLTYSCRSSQDCIINKHHRNRCQFCRLKKCLEMGMKMESVQSERKPFDVQREKPSNCAASTEKIYIRKDLRSPLIATPTFVADKDGARQTGLLDPGMLVNIQQPLIREDGTVLLATDSKAETSQGALGTLANVVTSLANLSESLNNGDTSEIQPEDQSASEITRAFDTLAKALNTTDSSSSPSLADGIDTSGGGSIHVISRDQSTPIIEVEGPLLSDTHVTFKLTMPSPMPEYLNVHYICESASRLLFLSMHRARSIPAFQGLGQDCNTSLVRACWNELFTLGLAQCAQVMSLSTILAAIVNHLQNSIQEDKLSGDRIKQVMEHIWKLQEFCNSMANWDIDGYEYAYLKAIVLFSPDHPGLTSTSQIEKFQEKAQMELQDYVQKTYSEDTYRLARILVRLPALRLMSSNITEELFFTGLIGNVSIDSIIPYILKMETAEYNGQITGVSL

SEQ ID NO: 9 - DNA, 2254 bp, Artificial Sequence
  Description of Artificial Sequence; note=synthetic construct
gaattcgccgctcccacctcggcgtctcgtctctcgcccgctgccccgcgagcccgcggcccccggctcccgccatccgccgacaccgggagcccgggctccccgcgccctgccctccgcgccgggggccgcccgccgcagacacgggacccgcttcgaggccgctttggcgcaaatcctgaggtaacacgtacacagacctctcggccggaatctccagggatgactagcccctccccacgcgtccagataatctccaccgactctgctgtagcctcacctcagcgcattcagggctctgaacctgcctctggcccattgagtgttttcacatctttgaacaaagagaagattgtcacagaccagcagacaggacagaaaatccagatagtcaccgcagtggacgcctccggatcccccaaacagcagttcatcctgaccagcccagatggagctggaactgggaaggtgatcctggcttccccagagacatccagcgccaagcaactcatattcaccacctcagacaacctcgtccctggcaggatccagattgtcacggattctgcctctgtggagcgtttactggggaagacggacgtccagcggccccaggtggtagagtactgtgtggtctgtggcgacaaagcctccggccgtcactatggggctgtcagttgtgaaggttgcaaaggtttcttcaaaaggagtgtgaggaaaaatttgacctacagctgccggagcagccaagactgcatcatcaataaacatcaccggaaccgctgtcagttttgccggctgaaaaaatgcttagagatgggcatgaaaatggaatctgtgcagagtgaacggaagcccttcgatgtgcaacgggagaaaccaagcaattgtgctgcttcaactgagaaaatctatatccggaaagacctgagaagtcccctgatagctactcccacgtttgtggcagacaaagatggagcaagacaaacaggtcttcttgatccagggatgcttgtgaacatccagcagcctttgatacgtgaggatggtacagttctcctggccacggattctaaggctgaaacaagccagggagctctgggcacactggcaaatgtagtgacctcccttgccaacctaagtgaatctttgaacaacggtgacacttcagaaatccagccagaggaccagtctgcaagtgagataactcgggcatttgataccttagctaaagcacttaataccacagacagctcctcttctccaagcttggcagatgggatagacaccagtggaggagggagcatccacgtcatcagcagagaccagtcgacacccatcattgaggttgaaggccccctcctttcagacacacacgtcacatttaagctaacaatgcccagtccaatgccagagtacctcaacgtgcactacatctgtgagtctgcatcccgtctgcttttcctctcaatgcaccgggcaaggtcaatcccagcctttcagggacttggacaggactgcaacaccagccttgtgcgggcctgctggaatgagctcttcaccctcggcctggcccagtgtgcccaggtcatgagtctctccaccatcctggctgccattgtcaaccacctgcagaacagcatccaggaagataaactttctggtgaccggataaagcaagtcatggagcacatctggaagctgcaggagttctgtaacagcatggcgaactgggatatagatggctatgagtatgcataccttaaagctatagttctctttagccccgatcatccaggtttgaccagcacaagccagattgaaaaattccaagaaaaggcacagatggagttgcaggactatgttcagaaaacctactcagaagacacctaccgattggcccggatcctcgttcgcctgccggcactcaggctgatgagctccaacataacagaagaacttttttttactggtctcattggcaatgtttcgatagacagcataatcccctacatcctcaagatggagacagcagagtataatggccagatcaccggagtcagtctatagcgcaaaccacacacctgccaaggagcaacagaatccttccaggaccgttcacatacaaagaaaagtagtggtattttggtatgtgcaaatatttccatatgttagccatttcctgtctggtttctccttatctgttaatcccagacaatagcaattaaaagactagtagggatcctttcctgac

SEQ ID NO: 10 - DNA, 15 bp, Artificial Sequence
  Description of Artificial Sequence; note=synthetic construct
agccctactaattac

SEQ ID NO: 11 - DNA, 13 bp, Artificial Sequence
  Description of Artificial Sequence; note=synthetic construct
tctttgcaggatt

SEQ ID NO: 12 - DNA, 14 bp, Artificial Sequence
  Description of Artificial Sequence; note=synthetic construct
aagcaaatccatat

SEQ ID NO: 13 - DNA, 15 bp, Artificial Sequence
  Description of Artificial Sequence; note=synthetic construct
ccctccttctaacat

SEQ ID NO: 14 - DNA, 15 bp, Artificial Sequence
  Description of Artificial Sequence; note=synthetic construct
ccctccttccaacat

SEQ ID NO: 15 - DNA, 21 bp, Artificial Sequence
  Description of Artificial Sequence; note=synthetic construct
caataataggattcccaatcg

SEQ ID NO: 16 - DNA, 21 bp, Artificial Sequence
  Description of Artificial Sequence; note=synthetic construct
cagtattaggattcctaatcg

SEQ ID NO: 17 - DNA, 21 bp, Artificial Sequence
  Description of Artificial Sequence; note=synthetic construct
tacccctattaatatttttcc

SEQ ID NO: 18 - DNA, 21 bp, Artificial Sequence
  Description of Artificial Sequence; note=synthetic construct
taaccctagtattatttttcc

SEQ ID NO: 19 - DNA, 20 bp, Artificial Sequence
  Description of Artificial Sequence; note=synthetic construct
actaatcctagccctagccc

SEQ ID NO: 20 - DNA, 20 bp, Artificial Sequence
  Description of Artificial Sequence; note=synthetic construct
aataaccctacccctagccc

SEQ ID NO: 21 - DNA, 30 bp, Artificial Sequence
  Description of Artificial Sequence; note=synthetic construct
ctatcactcacactagcattaagtctatga

SEQ ID NO: 22 - DNA, 30 bp, Artificial Sequence
  Description of Artificial Sequence; note=synthetic construct
ctacccctaacactagcattatgtatgtga

SEQ ID NO: 23 - DNA, 16 bp, Artificial Sequence
  Description of Artificial Sequence; note=synthetic construct
cctgagccctaataat

SEQ ID NO: 24 - DNA, 13 bp, Artificial Sequence
  Description of Artificial Sequence; note=synthetic construct
acctccctcacca

SEQ ID NO: 25 - DNA, 23 bp, Artificial Sequence
  Description of Artificial Sequence; note=synthetic construct
ggttgcctgattgtggatctgtg

SEQ ID NO: 26 - DNA, 24 bp, Artificial Sequence
  Description of Artificial Sequence; note=synthetic construct
gctgactccttaatgaggggtgag

SEQ ID NO: 27 - DNA, 24 bp, Artificial Sequence
  Description of Artificial Sequence; note=synthetic construct
acagcccgattattttgctactcc

SEQ ID NO: 28 - DNA, 24 bp, Artificial Sequence
  Description of Artificial Sequence; note=synthetic construct
tttcttcaaggtagttgggttccc

SEQ ID NO: 29 - DNA, 23 bp, Artificial Sequence
  Description of Artificial Sequence; note=synthetic construct
tgaacggtgatggcatctgaatg

SEQ ID NO: 30 - DNA, 24 bp, Artificial Sequence
  Description of Artificial Sequence; note=synthetic construct
ttttccttcctgcatggttctttg

SEQ ID NO: 31 - DNA, 23 bp, Artificial Sequence
  Description of Artificial Sequence; note=synthetic construct
tgtgtgggtgtcagatggttgtc

SEQ ID NO: 32 - DNA, 20 bp, Artificial Sequence
  Description of Artificial Sequence; note=synthetic construct
ttatttcggtgccctgatgg